(19) United States
(12) Patent Application Publication
(10) Pub. No.: US 2025/0352103 A1
(43) Pub. Date: Nov. 20, 2025

(54) CONTACTLESS STRESS MONITORING USING WIRELESS SIGNALS

(71) Applicants: Massachusetts Institute of Technology, Cambridge, MA (US); The Board of Trustees of the University of Illinois, Urbana, IL (US)

(72) Inventors: Fadel Adib, Cambridge, MA (US); Unsoo Ha, Cambridge, MA (US); Seyedsohrab Madani, Watertown, MA (US)

(73) Assignees: Massachusetts Institute of Technology, Cambridge, MA (US); The Board of Trustees of the University of Illinois, Urbana, IL (US)

(21) Appl. No.: 18/686,104

(22) PCT Filed: Oct. 31, 2022
(86) PCT No.: PCT/US2022/048374

§ 371 Date: Feb. 23, 2024

Related U.S. Application Data
(60) Provisional application No. 63/276,889, filed on Nov. 8, 2021.

(51) Int. Cl. A61B 5/16; A61B 5/00; A61B 5/0205; A61B 5/024; A61B 5/0245; A61B 5/0255; A61B 5/05; A61B 5/08; A61B 5/11; A61B 5/113; A61B 5/346; G16H 50/70

(52) U.S. Cl. CPC A61B 5/165; A61B 5/0205; A61B 5/02416; A61B 5/0245; A61B 5/0255; A61B 5/05; A61B 5/08; A61B 5/1126; A61B 5/113; A61B 5/346; A61B 5/7246; A61B 5/7267; G16H 50/70; A61B 2562/046

(57) ABSTRACT

According to one aspect of the disclosure, a method for measuring stress of a subject includes: transmitting, by a sensor, a wireless signal within an environment comprising the subject: measuring reflections of the wireless signal to generate a physiological signal responsive to changes in distance between the subject and the sensor over time: processing the physiological signal to extract feature data of the subject; and providing the feature data as input to a stress classification network to determine a stress level of the subject.

### CROSS-REFERENCE TO RELATED APPLICATIONS

This application claims the benefit under 35 U.S.C. § 119 of U.S. Provisional Patent Application No. 63/276,889 filed on Nov. 8, 2021, which is hereby incorporated by reference herein in its entirety.

### STATEMENT REGARDING FEDERALLY SPONSORED RESEARCH

This invention was made with government support under CNS-1844280 awarded by the National Science Foundation and N00014-19-1-2325 awarded by U.S. Navy, Office of Naval Research. The government has certain rights in the invention.

### BACKGROUND

Stress plays a critical role in our lives, impacting our productivity and our long-term physiological and psychological well-being. We all experience stress during our daily lives: while working on a deadline, preparing for an event, worrying about our children, or thinking about our futures. While moderate stress may boost our productivity, chronic stress—or prolonged exposure to acute stress—has detrimental outcomes. It accelerates aging at the cellular level and promotes an earlier onset of age-related diseases such as diabetes, Alzheimer's, and high blood pressure. Clinical studies have also shown that chronic stress is correlated with increased risk of depression, fatigue, anxiety, and insomnia.

Studies have motivated the development of stress monitoring solutions to better understand stress, its impact on productivity and teamwork, and help users adapt their habits toward more sustainable stress levels. In particular, there is a need for continuous and fully-automated stress monitoring. Such monitoring can provide doctors with information to better manage patient conditions; it can also help us modulate our own stress levels by incorporating meditation or adapting our daily activities to improve our well-being and productivity.

### SUMMARY

Today's stress monitoring solutions remain obtrusive, requiring active user participation (e.g., self-reporting), interfering with people's daily activities, and often adding more burden to users looking to reduce their stress. The most standard approach relies on self-reporting, where users log their stress levels (e.g., low, medium, or high) in a journal or on a smartphone, making this approach obtrusive. Another common approach for one-shot, acute stress measurement is to extract the cortisol hormone from saliva samples. This approach is not suitable for long-term monitoring, as it relies on precise lab equipment, the subject's willingness to frequently provide saliva samples, and an expert's intervention to extract the hormone concentration.

Recognizing these limitations, researchers have looked into inferring stress levels from wearables that monitor stress-related biomarkers such as breathing, heart rate variability (HRV), and skin conductivity. These solutions fall into two major categories, which trade off comfort for accuracy. The first can accurately extract stress-related metrics but require bulky setups that can be burdensome for everyday use (e.g., chest bands that measure HRV or shirts that measure muscle activity to infer stress). The second category uses wristbands that can infer stress from HRV measurements. These approaches are more comfortable for everyday use, but they have lower accuracy than chest bands due to the constant movement of a user's wrist, which has driven medical researchers to adopt protocols that use chest bands over wristbands for HRV measurements. Moreover, all of these approaches require users to actively participate and wear devices, which may be uncomfortable for various sections of the population.

There is a need for monitoring solutions that monitor a user in a passive manner and without interfering with the user's daily activities. Disclosed herein are systems and techniques for monitoring a user's stress passively using wireless signals.

The past two decades have witnessed an increased interest in stress monitoring to prevent, diagnose, and even treat related diseases. The standard approach for stress monitoring relies on extracting cortisol from saliva samples or requiring users to complete questionnaires about their stress level. Because these methods are obtrusive and inconvenient for long-term stress monitoring, researchers have looked into a variety of alternative methods for inferring stress.

Recent studies have used a variety of wearables such as smart belts, shirts, smart watches, and skin-attachable sensors for stress monitoring. These wearables typically infer stress by measuring biomarkers such as HRV, electrodermal activity, and respiration.

This body of work has a number of limitations. First, these methods still require attachment to the person's skin and/or body. As a result, certain population (e.g., children, elderly) may not be comfortable with wearing these devices for a long time, and a recent survey reported that around 15 to 35 percent of the respondents would not be willing to continuously wear a stress monitoring device. Furthermore, these wearables typically exhibit a trade-off between accuracy and comfort (more bulky devices are more accurate, but less comfortable), with certain devices requiring inconvenient skin preparation; for example, some existing devices require that any hair be removed from the area before the sensor is attached. Aside from the potential inconvenience of wearing these devices, the user must remember to recharge them on a daily basis.

In contrast to such prior systems and techniques, systems and techniques disclosed herein do not require any contact with the user's body or any cooperation from the user (e.g., re-charging). More importantly, they open up new use cases that require location-specific monitoring. For example, disclosed systems and techniques can be incorporated into smart devices (like screens or kiosks) to infer when a user interacting with them is stressed and adapt to their stress level, or it can be installed in offices or classrooms to help monitor work stress of employees and/or students. Of note, GPS-enabled wearables generally cannot enable location-specific monitoring in indoor environments because GPS accuracy is poor indoors.

Camera-based methods have also been proposed to assess stress level by sensing visual cues such as a subject's head movements, blink rate, and pupil size variation. Some methods also use videos to estimate the HRV of the subject in order to infer their stress level. However, these methods involve capturing high resolution video of the subject to capture the minute changes corresponding to small variations in the user's face, which raises privacy concerns. Additionally, these methods are sensitive to lighting conditions and do not work in the dark. In contrast, systems and technique disclosed here are much less privacy intrusive and work in the dark.

Researchers have also considered smartphones to monitor stress by analyzing user behavior These analyses include speech analysis, location behavior, phone call and text patterns, and user personal traits. However, these studies have reported lower (70% or less) accuracy and reliability than bio-markers in inferring a user's stress, and raise privacy concerns for their users. Recent work has also leveraged smartphones for photoplethysmogram (PPG)-based HRV extraction, which is then used to assess the mental health of the subject. However, this approach shares the same challenges as contact-based methods since the user needs to put their finger directly on the smartphone camera and keep it there for the full duration of the experiment.

RF-based sensing is an emerging field which uses the reflections of RF (Radio Frequency) signals off the human body to track human movements, postures, and vital sign. Past systems that measure heartbeats using RF signals can be divided into two main categories.

The first category extracts the average heart rate from measurements collected over 1-2 minutes. This category of systems can work with quasi-random user movements because it averages out these movements over the measurement period. However, it cannot extract individual heartbeats, which are of the order of 1 second; as a result, this category of systems cannot measure HRV at the level of individual heartbeats, which is required for stress monitoring.

The second category can extract individual heartbeats and measure the HRV, but can only work in controlled lab environments. Specifically, these systems require users to sit or lie down in a specific position/orientation with respect to the sensing device, remain fully static for the measurement duration, and often require users to hold their breath. In the absence of such controlled lab setups, state-of-the-art systems (like EQ-Radio and RF-SCG (seismocardiograph)) incur errors that are ten to twenty-five times larger than those incurred by the systems and techniques disclosed herein, making them incapable of inferring a user's stress level, as the inventors of this patent application have demonstrated empirically. As a result, using these existing systems for stress monitoring would require users to stop their daily activities in order to actively take a measurement. More importantly, they would make the user aware of being monitored, thus inducing the well-known subject-expectancy effect where the experimental setup biases the results.

Systems and techniques disclosed herein improve on these prior systems to deliver the first fully automated, passive system for monitoring stress by extracting HRV without imposing unnatural requirements on the user. Disclosed embodiments include: (1) passive stress monitoring system that can infer a user's stress levels from wireless signals; (2), a novel machine learning network that can extract HRV from wireless reflections without requiring users to remain static during the monitoring phase; and (3) a framework for extracting physiological and motion-based features from wireless signals for stress monitoring.

Systems and techniques disclosed herein provide for contactless stress monitoring that can infer user stress levels from wireless signals. Embodiments have been shown to work correctly without requiring any contact with the user's body, or the user being aware of the system. By automatically and opportunistically extracting vital signs and other stress-related features from a nearby user and designing a novel pipeline for extracting stress-related features from wireless signals, disclosed embodiments enable long-term monitoring of user stress levels.

In comparison to existing approaches for stress monitoring—which either solicit user input or require users to wear on-body sensors (e.g., ECG, GSR, PPG)—systems and techniques disclosed herein offer a more seamless, transparent, and convenient modality for long-term stress monitoring. They open up opportunities for monitoring stress in workplaces or academic environments, where the sensor can be placed in a room or on a desk to monitor nearby users. Such deployments can help boost productivity and performance and reduce burnout, as well as inform intervention mechanisms to support student and worker mental health and well-being. Embodiments of the present disclosure can be incorporated into smart devices (e.g., screens, kiosks, TVs, smart home assistants) to help understand and respond to user stress levels. For example, they can be used in studies for user experience evaluation or in everyday environments to enable interactive capabilities (adapt their tone, colors, etc.) based on user stress levels. Additionally, embodiments of the present disclosure can be used to assess stress during sleep and help improve sleep quality. Beyond smart devices and smart environments, systems and techniques described herein can be incorporated in social robots to help them asses user stress, improve interventions for aging and child learning, and support long-term psychological well-being.

More generally, systems and techniques disclosed herein pave the way towards transparent and ubiquitous stress monitoring systems, with applications spanning smart homes, human-computer interaction, and mental health and well-being.

According to one aspect of the disclosure, a method for measuring stress of a subject can include: transmitting, by a sensor, a wireless signal within an environment comprising the subject; measuring reflections of the wireless signal to generate a physiological signal responsive to changes in distance between the subject and the sensor over time; processing the physiological signal to extract feature data of the subject; and providing the feature data as input to a stress classification network to determine a stress level of the subject.

In some embodiments, the feature data can include data representing respiration of the subject. In some embodiments, the processing of the physiological signal can include: filtering the physiological signal using a band-pass filter to generate a respiration signal responsive to respiration of the subject; and identifying local maxima and minima of the respiration signal to extract the data representing respiration of the subject.

In some embodiments, the feature data can include data representing heartbeats of the subject. In some embodiments, processing of the physiological signal can include: dividing the physiological signal into a plurality of time-domain segments; extracting a plurality of time-domain features from the physiological signal by processing individual ones of the plurality of time-domain segments using a feature extraction network; generating a self-similarity matrix (SSM) by cross-correlating the plurality of time-domain features; and using the SSM to extract the data representing heartbeats of the subject.

In some embodiments, the feature data can include data representing body movements of the subject.

In some embodiments, the feature data can include: data representing respiration of the subject; data representing heartbeats of the subject; and data representing body movements of the subject.

In some embodiments, the transmitting of the wireless signal can include transmitting a millimeter wave signal. In some embodiments, the transmitting of the wireless signal can include transmitting a Frequency-Modulated Continuous Wave (FMCW) wireless signal. In some embodiments, the transmitting of the wireless signal can include transmitting the wireless signal via an antenna array of the sensor. In some embodiments, the environment can include multiple subjects, and the method further can further include beamforming the wireless signal in a direction of the subject.

According to another aspect of the disclosure, a method for extracting heartbeats intervals from a noisy time-domain physiological signal can include: extracting a plurality of time-domain features from the physiological signal using a feature extraction network; generating a self-similarity matrix (SSM) by cross-correlating the plurality of time-domain features; processing the SSM using a heartbeat extraction network to: identify heartbeat patterns within the physiological signal; and extract the heartbeat intervals using the identified heartbeat patterns.

In some embodiments, the method can further include: measuring, by a sensor, reflections of a wireless signal to generate the physiological signal responsive to changes in distance between a subject and the sensor over time. In some embodiments, the physiological signal can be received from an electrode. In some embodiments, the physiological signal can be received from a wearable device. In some embodiments, the physiological signal may correspond to an electrocardiogram (ECG) signal. In some embodiments, the physiological signal may correspond to a photoplethysmography (PPG) signal. In some embodiments, the physiological signal can correspond to a seismocardiograph (SCG) signal.

In some embodiments, the extracting of the plurality of time-domain features from the physiological signal can include: dividing the physiological signal into a plurality of time-domain segments; and extracting the plurality of time-domain features from the physiological signal by processing individual ones of the plurality of time-domain segments using a feature extraction network.

In some embodiments, the heartbeat extraction network can include a two-dimensional (2D) convolutional neural network (CNN). In some embodiments, the CNN can be trained to classify individual ones of the plurality of time-domain features as corresponding to a heartbeat or not correspond to a heartbeat. In some embodiments, the method may further include: generating a set of indices indicating which segments of the physiological signal correspond to heartbeats based on the classifications, wherein the heartbeat extraction network extracts the heartbeat intervals using the set of indices.

According to another aspect of the disclosure, a method for measuring stress of a subject can include: receiving one or more time-domain signals responsive to the subject; extracting feature data from the one or more time-domain signals, the feature data including at least: data representing vital signs of the subject, and data representing body movements of the subject; and providing the feature data as input to a stress classification network to determine a stress level of the subject.

In some embodiments, the receiving of the one or more time-domain signals can include receiving a physiological signal, and the method can further include: measuring, by a sensor, reflections of a wireless signal to generate the physiological signal responsive to changes in distance between the subject and the sensor over time. In some embodiments, the receiving of the one or more time-domain signals can include receiving a signal from a wearable device associated with the subject. In some embodiments, the receiving of the one or more time-domain signals can include receiving a signal from an electrode associated with the subject. In some embodiments, the receiving of the one or more time-domain signals includes receiving a signal from a camera directed at the subject.

In some embodiments, the data representing vital signs of the subject can at least includes data representing respiration of the subject. In some embodiments, the data representing vital signs of the subject can at least includes data representing heartbeats of the subject. In some embodiments, the stress classification network can be trained using datasets of time-domain signals from subjects under stress. In some embodiments, the stress classification network can include a random forest classifier. In some embodiments, the stress classification network can include a neural network.

It should be appreciated that individual elements of different embodiments described herein may be combined to form other embodiments not specifically set forth above. Various elements, which are described in the context of a single embodiment, may also be provided separately or in any suitable sub-combination. It should also be appreciated that other embodiments not specifically described herein are also within the scope of the following claims.

### BRIEF DESCRIPTION OF THE DRAWINGS

The manner of making and using the disclosed subject matter may be appreciated by reference to the detailed description in connection with the drawings, in which like reference numerals identify like elements.

FIG. 1A is a pictorial diagram illustrating passive stress monitoring using wireless signals, according to some embodiments.

FIG. 1B is a block diagram showing an example of a system for passive stress monitoring using wireless signals, according to some embodiments.

FIG. 1C is a schematic diagram illustrating processing that can occur within the system of FIG. 1B, according to some embodiments.

FIG. 2 is a schematic diagram of showing another example of a system for passive stress monitoring, according to some embodiments.

FIG. 2A is a schematic diagram showing addition details of the system of FIG. 2, according to some embodiments.

FIG. 3A is a waveform diagram illustrating a fixed heartbeat pattern from an electrocardiogram (ECG) signal.

FIG. 3B is a waveform diagram illustrating heartbeats from a captured wireless signal, according to some embodiments.

FIG. 3C is a waveform diagram illustrating motion artifact caused by user movements, according to some embodiments.

FIGS. 4A and 4B show examples of self-similarity matrices (SSMs) that can be generated from wireless reflections, according to some embodiments.

FIGS. 5A and 5B show examples of SSMs that can be generated from wireless reflections are corrupted by body movements, according to some embodiments.

FIGS. 6, 6A, and 6B collectively illustrate a self-similarity-based perception network that can be used to isolate individual heartbeat features, according to some embodiments.

FIG. 7A is a waveform illustrating an idealized IBI time series.

FIG. 7B is a waveform illustrating sparsity in an IBI time series due to discarding segments with motion contamination, according to some embodiments.

FIGS. 7C and 7D illustrate a sparsity simulation module that can be used to aid passive stress monitoring using wireless signals, according to some embodiments.

FIG. 7E shows an example of a network that can be used in conjunction within the sparsity simulation module of FIGS. 7C and 7D, according to some embodiments.

FIG. 7F shows an example of an indices matrix that can be generated for input to the network of FIG. 7E, according to some embodiments.

FIGS. 8A and 8B are a series of plots illustrating the extraction of physiological and motion-based features for stress classification, according to some embodiments.

FIG. 9 shows a series of waveforms illustrating non-linear data augmentation that can be used to train a neural network, according to some embodiments.

FIGS. 10-12 are flow diagrams showing examples of processes for monitoring stress, according to some embodiments.

FIG. 13 is block diagram of a processing device on which methods and processes disclosed herein can be implemented, according to some embodiments of the disclosure.

The drawings are not necessarily to scale, or inclusive of all elements of a system, emphasis instead generally being placed upon illustrating the concepts, structures, and techniques sought to be protected herein.

### DETAILED DESCRIPTION

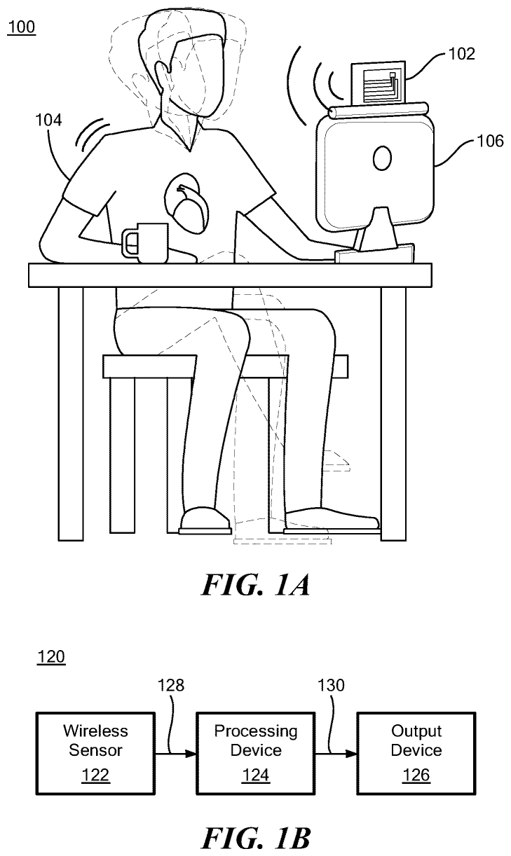

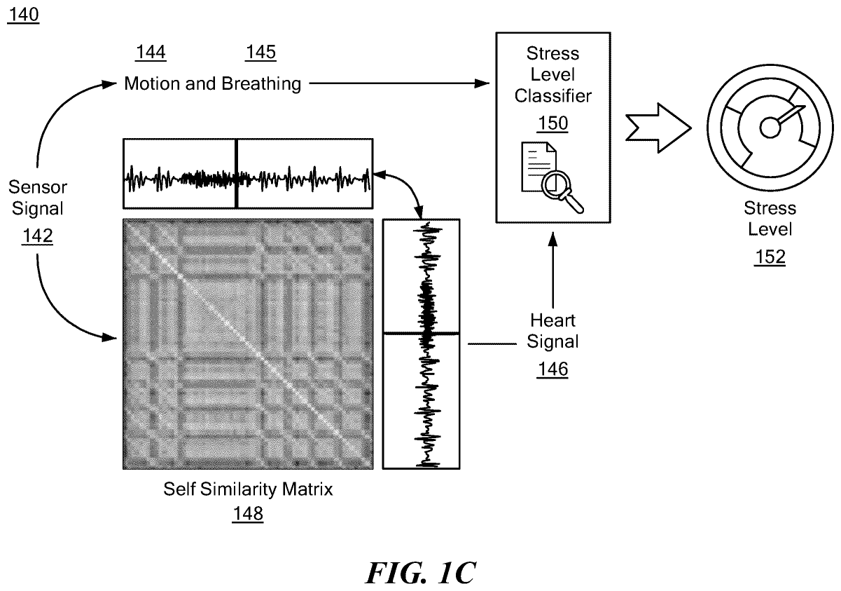

FIG. 1A illustrates passive stress monitoring using wireless signals, according to the present disclosure. A monitoring device 102 can be arranged to monitor stress of a user (e.g., human subject) 104 within an environment 100, such as an office, bedroom, living room, automobile, etc. For example, monitoring device 102 can be installed on a desk or near a couch to monitor a nearby user's stress levels. It works by continuously transmitting ultra-low-power wireless signals that reflect off the user's body and capturing these reflections in order to infer the user's stress level using processing techniques described herein. In some embodiments, monitoring device 102 may interface with a host computer 106 to perform some/all of said processing and to output an indicating of the user's stress level (e.g., via a display device of host computer 106). In other embodiments, monitoring device 102 may be configured to perform said processing itself (e.g., monitoring device 102 may be a standalone stress monitoring device).

While the user 104 in FIG. 1A is shown seated at a desk, the passive stress monitoring systems and techniques disclosed herein may be used accurately determine a user's stress level even as the user moves around their environment 100, leaving and returning to the radio range of the monitoring device 102 and, moreover, while other people/objects freely move around in the background.

FIG. 1B shows a system 120 for passive stress monitoring using wireless signals, according to some embodiments. System 120 is a passive stress monitoring system that relies on wireless signals. The system uses a wireless device that can sit on a user's desk or near their couch such as shown in FIG. 1A. The device continuously can send an ultra-low-power RF signal in the millimeter-wave band and captures its reflection. It analyzes these reflections over time in order to detect the nearest user and infer the user's stress level.

Illustrative system 120 includes a wireless sensor 122, a processing device 124, and an output device 126. In some embodiments, the components 122, 124, 126 can be integrated into a single, standalone device. In other embodiments, different components 122, 124, 126 may be integrated into different devices. For example, wireless sensor 122 may and processing device 124 may be separate devices that communicate via a wired or wireless link (e.g., USB, Ethernet, Bluetooth, Wi-Fi, or other type of link). In some embodiments, wireless sensor 122 may correspond to monitoring device 102 of FIG. 1A, and both processing device 124 and output device 126 may correspond to host computer 106 of FIG. 1A.

Wireless sensor 122 can be configured to transmit wireless signals within an environment comprising a user (e.g., user 104 of FIG. 1A) and to measure reflections of the wireless signals to generate a signal 128 having a phase responsive to a distance between objects in the environment (e.g., a user's body) and the sensor 122. Wireless sensor 122 can transmit wireless signals on a continuous basis, such that that phase of signal 128 varies continuously over time (e.g., in response to movements of the user).

In some embodiments, wireless sensor 122 can include a millimeter-wave radar (e.g., a radar operating within the frequency range of 30-300 GHz) and, in some cases, may be provided as an off-the-shelf millimeter-wave sensing board, such as the IWR1443BOOST board/module from TEXAS INSTRUMENTS. In some embodiments, wireless sensor 122 can be configured to transmit a frequency-modulated continuous-wave (FMCW) radar signal having a selected center frequency (e.g., 77 GHz) and bandwidth (e.g., 4 GHZ). Wireless sensor 122 can include one or more antennas for transmitting and receiving wireless signals and, in some cases, may include one or more array antennas that can be used for beamforming. For example, wireless sensor 122 may include two linear array antennas for beamforming: horizontal (with 3-dB beam-width of ±28°) and vertical/elevation (with 3-dB beam-width of ±14°), implemented as a 3-switched-transmitter and 4-receiver system. In some embodiments, wireless sensor 122 may correspond to a millimeter-wave radar provided within an existing consumer electronic device, such as the GOOGLE NEST HUB.

Processing device 124 may correspond to a general purpose computer or an application-specific integrated circuit (ASIC) configured to process the sensor signal 128 generated by the wireless sensor 122 using various techniques disclosed. In some embodiments, wireless sensor 122 may provide a digital output signal 128 that can be directly processed by a digital circuitry of processing device 124. In other embodiments, wireless sensor 122 may provide an analog output signal 128 and processing device 124 may include an analog-to-digital (ADC) converter for converting the sensor signal 128 into a digital signal for processing.

In response to the sensor signal 128, processing device 124 can generate data 130 indicating a stress level of the user which is provided to output device 126. Briefly, processing device 124 can process sensor signal 128 to generate a time-domain physiological signal responsive to changes in distance between the subject and the sensor 122 over time, process the physiological signal to extract feature data of the user, and provide the feature data as input to a stress classification network to determine data 130 representing, for example, a stress level of the user. These and additional processing techniques that can be performed by processing device 124 are described in detail below.

Processing device 124 can run one or more software packages to receive and process the sensor signal 128. For example, processing device 124 may receive data captured by wireless sensor 122 using the MMWAVE STUDIO software developed by TEXAS INSTRUMENTS. Similar software may also be used to configure the parameters of wireless sensor 122. As another example, processing device 124 can perform certain signal processing (e.g., beamforming, filtering, etc.) using numeric computing software, such as MATLAB. As discussed below, processing device 124 can employee one or more neural networks to classify individual time-domain features extracted from sensor signal 128 and to determine a stress level of the subject based on such features. Thus, in some embodiments, processing device 124 may run a ML toolkit such as TENSORFLOW, PYTORCH, etc. In some embodiments, a toolkit such as PSYTOOLKIT may be used to design a dedicated stress elicitation software that can be run on processing device 124.

Output device 126 can use the data 130 generated by processing device 124 to output a stress level of a user. In some embodiments, output device 126 may correspond to a display device (e.g., a monitor or touchscreen) configured to display a graphical representation of the user's stress level. For example, data 130 may encode the stress level as a numeric value within a predetermine range (e.g., between 0 and 10, between 0 and 100, etc.) and output device 126 may display the stress level on a graphical representation of a gauge/scale having the same range. In some embodiments, output device can include a database or other storage means for storing stress level data for a given user, or group of users, along with a user interface (UI) for retrieving such stored data. For a given user, output device 126 may store stress level data at different points in time to track the user's stress over days, weeks, months, years and provide a UI for visualization historical data trends. In some embodiments, output device 126 may correspond to an external system that tracks stress levels for groups of people, such as an external computer system associated with a health care provider, a research institution, etc. In this case, processing device 124 and output device 126 may communicate over a computer network such as the Internet.

FIG. 1C illustrates, at a high-level, processing 140 that can be used to infer a user's stress level from captured wireless signals. Some or all of the processing 140 illustrated in FIG. 1C may be implemented and executed by processing device 124 of FIG. 1B. A sensor signal 142 may processed to extract various feature data of a user, such as feature data 144 representing body movements of the user, feature data 145 representing respiration of the user, and feature data 146 representing heartbeats of the user. The feature data 144, 145, 146 may be providing as input to a stress classification network 150 to determine a stress level 152 of the user.

The concepts, structures, and techniques sought to be protected herein may be applied to non-human subjects, such as other mammals. It is known that stress levels can be inferred for other mammals using one or more of the same features used for human subjects, such as the heart rate variability, respiration, and/or motion features utilized herein. Thus, for example, the trainable networks described herein can be trained with data from human subjects or non-human subjects.

At the core of the design is a novel machine learning (ML) pipeline that can map captured wireless signals (or other similarly “noisy” signals) to stress levels. The pipeline extracts feature data for three key stress-correlated biometrics from such signals: respiration/breathing, heart-rate variability (HRV), and motion. Among these, HRV is particularly challenging because it requires sensing minute variations in the signal that arise from body movements triggered by heartbeats. Because heartbeat movements are very minute, they are easily masked by user movements as subtle as a shift in pose, nodding, shaking one's leg, or typing. As a result, unless the user is fully static, it is not possible to distinguish whether subtle changes in the signal (e.g., wireless reflections) are due to a heartbeat or due to a nod or an eye twitch let alone random noise, movements, or other users in the environment.

To overcome these challenges, embodiments of present disclosure identify and leverage temporally local self-similarities in the noisy signals and use them to zero in on a user's heartbeat. Specifically, rather than simply looking for subtle changes in the signals, disclosed systems look for similarities in these changes over short windows of time. Since a user's heartbeats are repetitive and the heart rate varies gradually over time, this approach allows the network to zero in on the heartbeats. This method is particularly powerful because it can also eliminate subtle random movements (e.g., nods, typing) and quasi-random movements (e.g., shaking legs). To learn temporally local self-similarities, embodiments of present disclosure opportunistically capture noisy signals over time and constructs a self-similarity matrix 148 similar to the one shown in FIG. 1C. As described further below, the matrix 148 compares time-shifted windows of signals to each other, and feeds them to a network that can learn similarity features due to heartbeats while eliminating changes arising from extraneous movements.

Additional processing can be built on top of this fundamental technique to deliver a fully-automated system for passive stress monitoring. Systems disclosed herein can automatically detect when a user is nearby and when they leave its sensing field. They may incorporate techniques that enable them to automatically identify and segment the variations in signals (e.g., wireless reflections) that arise from respiration and HRV, and mitigate the impact of extraneous movements and interference. Furthermore, rather than entirely discard measurements with motion artifacts, the user's body motion can be leveraged to boost its stress classification accuracy. This is because certain body movements (e.g., frequently shaking one's leg or stretching their neck) are correlated with stress levels. System architectures disclosed herein enable extracting and selecting physiological and motion-based features to train their learning models to infer a user's stress level.

The inventors have built a prototype passive stress monitoring system based on the structures and techniques disclosed herein using an off-the-shelf millimeter-wave sensing board, namely the TI IWR1443 module. They tested on 22 subjects of different ages and genders, across different homes, and during many different daily activities, as well as specific tasks designed to induce stress. Throughout the experiments, subjects were free to move around, leaving and returning the radio range of the sensor; moreover, other people freely moved around in the background. To obtain ground-truth measurements during the long-term studies, subjects were asked to fill out a standardized NASA-TLX form every 30 minutes.

The results of this experiment demonstrate that the systems and techniques disclosed herein can be used to passively and accurately classify among three standard levels of stress: low, moderate, and high. The prototype showed a median accuracy of 90.7% when the models were tested and trained on the same person (while a random guess is 33.3%). Moreover, it worked correctly even when it is tested on people it has never been trained on (and in new environments); in such scenarios its median accuracy remained over 84%. The inventors also demonstrated that the techniques disclosed herein can extract HRV's with very low error (median error <4 ms) even when subjects are free to perform daily activities; in contrast, the error of state-of-the-art HRV extraction algorithms from wireless signals increases to around 50 ms (i.e., 10× that demonstrated by the inventors) when subjects are allowed to perform daily activities, precluding the ability to use them for accurate and unobtrusive stress monitoring. Beyond obtaining spot-level stress checks, systems and techniques disclosed herein can be used track changes in a user's stress level over extended periods of time, paving way for future solutions that would allow users to monitor their stress levels and adapt their daily activities.

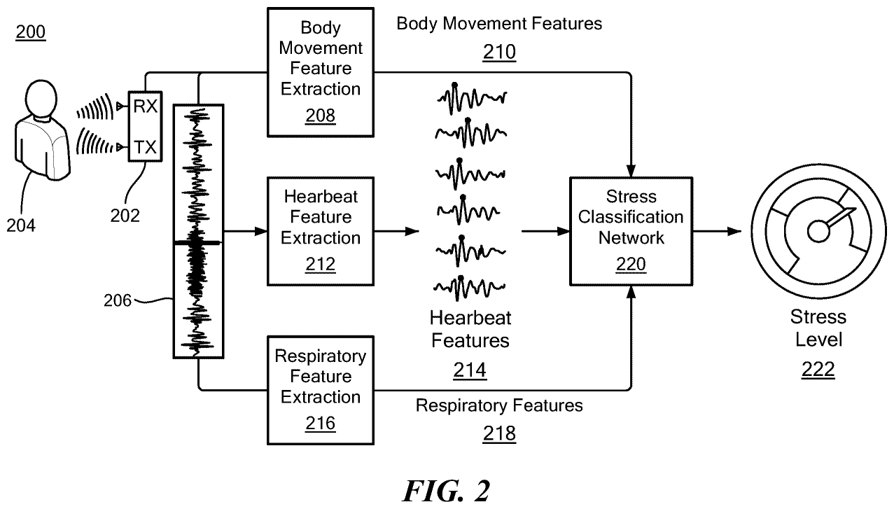

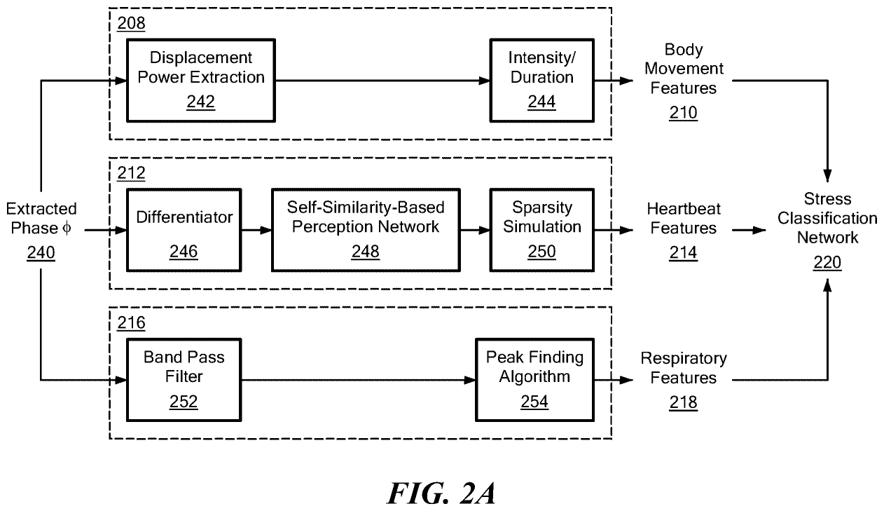

Turning to FIG. 2, an illustrative system 200 can be designed to include a wireless sensor 202 (e.g., a millimeter-wave FMCW radar) to extract wireless reflections off a body of a user 204. It filters these reflections to obtain a time-domain signal 206 that correspond to breathing cycles and micro-movements of the user 204. System 200 uses signal 206 to extract feature data within three different feature domains for stress level estimation. In particular, signal 206 can be fed into a body movement feature extraction pipeline 208 to extract body movement features 210, into a heartbeat extraction pipeline 212 to extract heartbeat features 214, and into a respiratory feature extraction pipeline 216 to extract respiratory features 218, as shown. Details of pipelines 208, 212, and 216 are described below in the context of FIG. 2A. Finally, the extracted features 210, 214, 218 are fed into a stress classification network 220 that infers a user's 204 stress level 222.

The illustrative system design of FIG. 2 can be described in terms of three stages. In the first stage, reflections are captured off a nearby user's 204 body. This stage can detect the presence of the user 204, determine when they leave and return to its sensing field (e.g., within approximately 3 m radius of the sensor), and eliminate the impact of other users and noise in the environment. It outputs a time-domain signal 206 corresponding to the user's 204 movements.

The second stage takes the time-domain signal 206 corresponding the user's movements and outputs heartbeat features 214 (e.g., hear rate variability data). This stage exploits a self-similarity matrix (SSM) to zero in on a user's heartbeats, and constructs a deep learning architecture that can robustly extract individual heartbeat intervals and eliminate extraneous movements. From the heartbeat intervals, system 200 can select and extracts stress-related heartbeat features 214. The body movement features 210 and respiratory features 218 can also be extracted in this second stage.

The third stage takes the extracted features 210, 214, 218 and uses the combined features to infer a user's 204 stress level 222.

Next, a detailed discussion of the general system design illustrated by FIG. 2 is provided.

The first step is to capture the wireless reflection of a nearby user's 204 movements. To do so, system 200 transmits a low-power RF signal (via sensor 202), measures its reflections, and filters them to zoom in on the nearby user 204. The main challenge in isolating the nearby user's reflections is that wireless signals not only reflect off that user's body, but also other objects in the environment, including furniture and other users.

To overcome these challenges, the illustrated system design builds on past systems that employ radar techniques in order to isolate the user's reflection and eliminate those arising from other objects in the environment. See, for example: (1) Fadel Adib, Chen-Yu Hsu, Hongzi Mao, Dina Katabi, and Frédo Durand, 2015, “Capturing the human figure through a wall,” “ACM Transactions on Graphics (TOG) 34, 6 (2015),” 1-13; and (2) Fadel Adib, Zach Kabelac, Dina Katabi, and Robert C Miller, 2014, “3d tracking via body radio reflections,” in “11th {USENIX} Symposium on Networked Systems Design and Implementation ({NSDI} 14),” 317-329, both of which references are hereby incorporated by reference in their entirety.

The techniques isolate reflections arriving from each 3D location in the environment by using a combination of FMCW radar and a 2D antenna array. Since different reflectors occupy different locations in 3D space, system 200 can use these techniques to isolate different reflectors into separate buckets. Subsequently, it eliminates all buckets with static reflections (e.g., furniture, walls) and identifies the nearest bucket that corresponds to a moving user. Of note, system 200 may be sensitive enough to pick up reflections that are varying due to other moving objects, e.g., fan or small pet. In such scenarios, it can eliminate these buckets using a technique described below.

Turning to FIG. 2A, once system 200 identifies the bucket that corresponds to a user, it extracts the phase 240 of the wireless signal in that bucket. The phase 240 captures any small changes in distance between the user's 204 body and the sensor's 202 antennas. Since both breathing and heartbeats are associated with variations in distance (due to chest movement or micro-vibrations on the surface of the body), the obtained time-domain signal 206 encodes movements corresponding to these vital signs. Mathematically, the phase is given by the following equation:

$$ϕ ( t ) = 2 π \frac{d ( t )}{λ} \qquad ( 1 )$$

where ϕ(t) is the phase of the received signal, λ is the wavelength of the signal, and d(t) denotes the distance over time between the device and the human body. That is, d(t) may correspond to signal 206 of FIG. 2.

FIG. 2A shows additional details (or “flow”) of the feature extraction pipelines 208, 212, and 214 and illustrates how system 200 uses three feature domains for stress level estimation. Each feature is extracted from the phase signal, processed through different pipelines, then fed into stress classification network 220 (e.g., a random forest classifier).

Heartbeat feature extraction pipeline 212 can process phase signal 240 to extract heartbeat features 214 of the user 204. Since heartbeats appear in d(t) by producing small mechanical vibrations on the user's chest area, system 200 can sense these vibrations through Eq. 1 by extracting the phase ϕ(t) 240 from the received signal and applying a double differentiator filter 246 such as described in more detail in the following publications: (1) Unsoo Ha, Salah Assana, and Fadel Adib. 2020, “Contactless seismocardiography via deep learning radars,” in “Proceedings of the 26th Annual International Conference on Mobile Computing and Networking,” 1-14; and (2) Mingmin Zhao, Fadel Adib, and Dina Katabi. 2016, “Emotion recognition using wireless signals” in “Proceedings of the 22nd Annual International Conference on Mobile Computing and Networking,” 95-108.

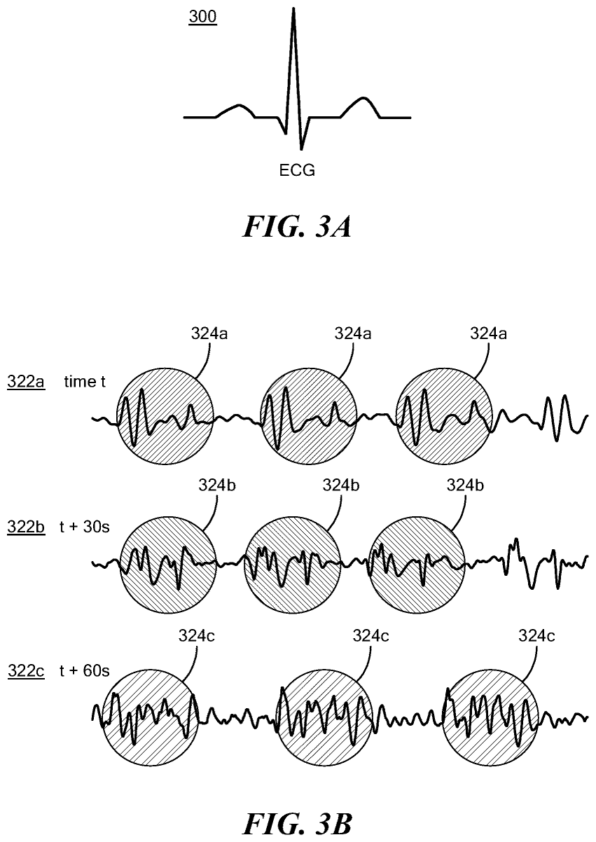

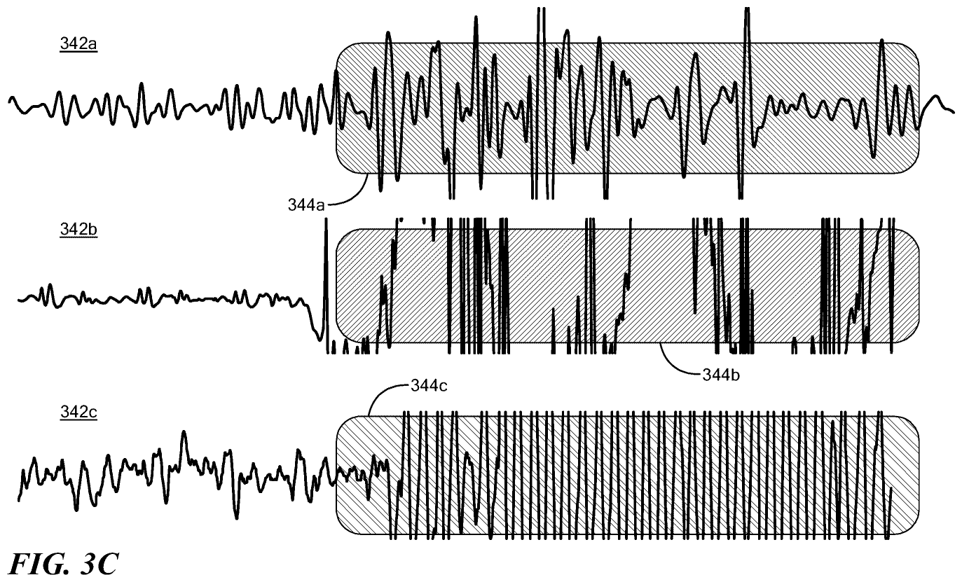

Now that system 200 has isolated the reflections from the user's 204 body, it proceeds to extracting individual heartbeats from this signal. To do so, it passes the time-domain signals output by differentiator filter 246 into a self-similarity-based perception network 248, a detailed description of which is given below in the context of FIGS. 3-6. Briefly, this network 248 exploits an SSM to zero in on a user's heartbeats, and constructs a deep learning architecture that can robustly extract individual heartbeat intervals and eliminate extraneous movements. Network 248 can output information about inter-beat-intervals (IBIs) between consecutive heartbeat patterns (IBIs are HRV values at the granularity of one heartbeat).

A challenge with IBI-based stress monitoring using contact-based methods (e.g., ECG, PPG) is that motion artifacts contaminate the signal, and the contaminated segments must be discarded prior to feeding them to the stress classifier. However, it is appreciated herein that adopting the same approach for passive stress monitoring using wireless signals may be undesirable for multiple reasons.

In the context of wireless sensing, simply discarding all segments with motion artifacts can negatively impact overall performance. This is because wireless signals are more sensitive to the human's motion than contact-based modalities. Specifically, the captured wireless reflection is affected by various kinds of body movements within the antennas' field-of-view (FoV), whereas wearables are affected only by the local body motions (e.g., moving the right hand while wearing a smartwatch on the left hand will not introduce noise). Moreover, since the signal reflections representing the mechanical movement of the heart are relatively weak, they are easily buried in other motion signals. Therefore, in a scenario where the user is moving, in order to recover accurate IBIs, it is necessary to discard those contaminated regions more often. Unfortunately, the discontinuity and missing values will distort the features and mislead the estimation, which would in turn reduce the stress classification accuracy.

To overcome this challenge, three techniques disclosed herein may be utilized.

First, while motion artifacts are typically considered harmful in standard contact-based modalities, it is appreciated herein that that motion itself contains meaningful information that can help in stress monitoring. For example, people under high stress typically exhibit specific body language such as frequently changing their body posture or shaking their foot/hand more often. Given its FoV, sensor 202, can sense these movements and they can be incorporated for use by stress classification network 220. Note that motion alone is typically not a sufficient feature to achieve high accuracy in stress classification. In particular, the subject may be moving due to a non-stress related reason. Hence, system 200 can use body movement features 210 as a contributing (rather than sole) feature in stress classification.

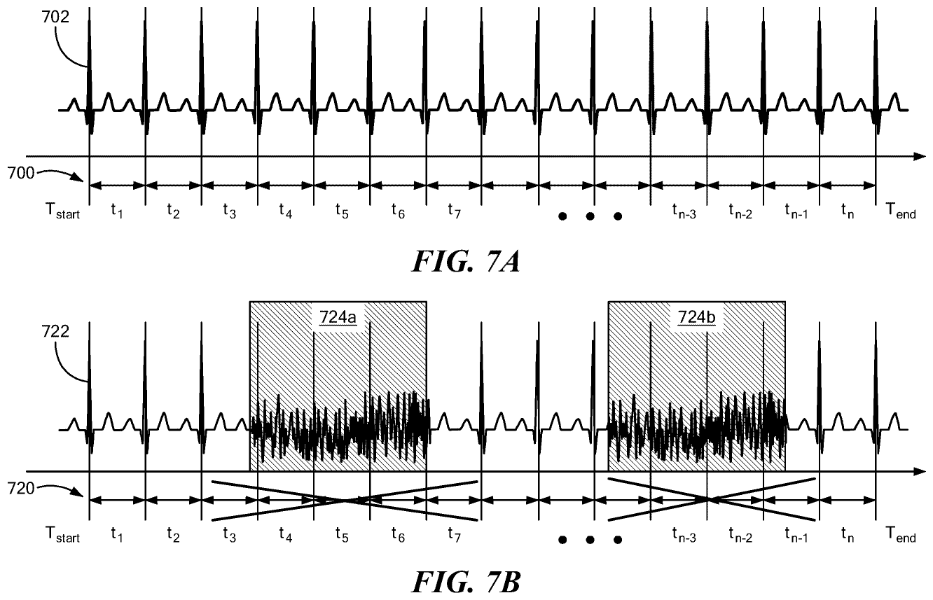

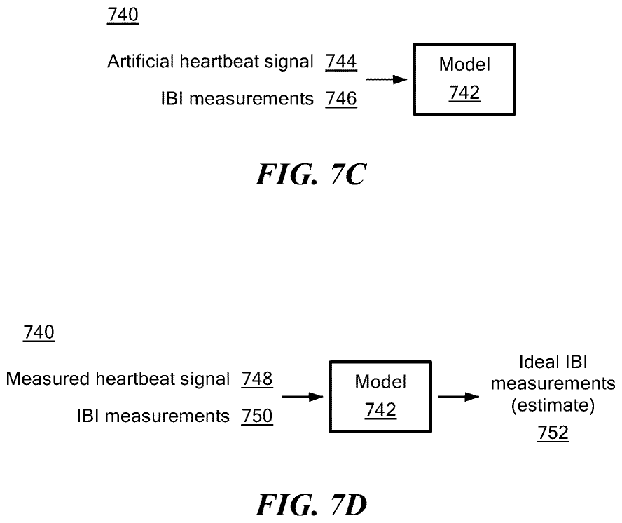

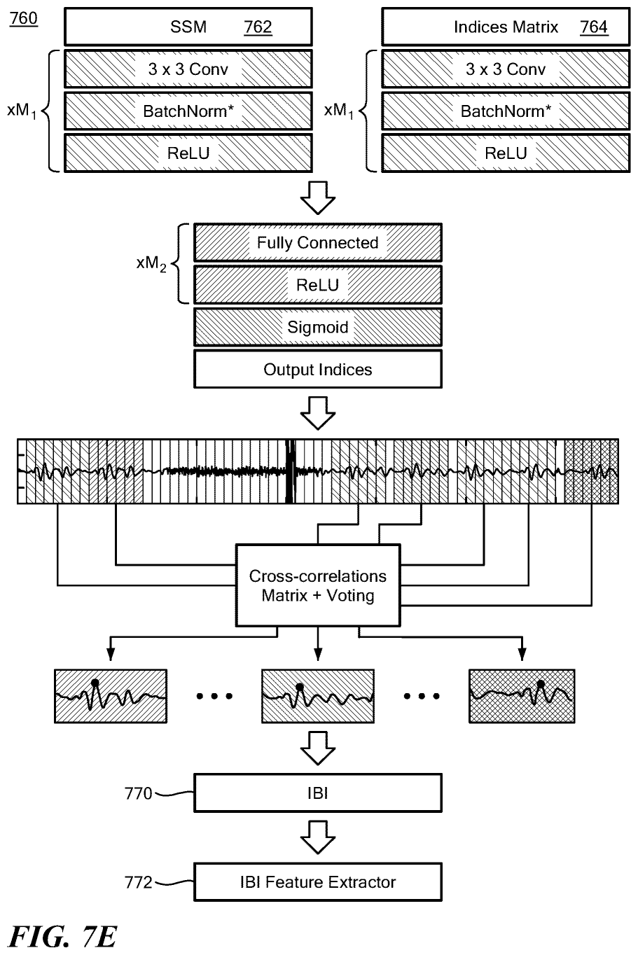

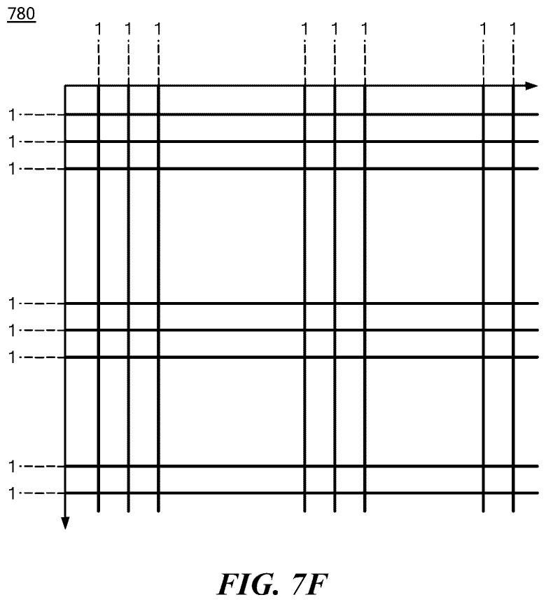

Second, while harnessing motion patterns can improve the accuracy of classification, heartbeat feature extraction pipeline 212 still needs to discard the corresponding time segments when extracting heartbeat features 214 to avoid errors in IBI estimation, but such discarding results in a sparse time series of IBI measurements. Thus, pipeline 212 can include a sparsity simulation module 250 that enables it to account for a sparse time series of IBI measurements. Details of sparsity simulation module 250 are provided below in the context of FIGS. 7A-7D.

Third, aside from IBI and motion patterns, system 200 also extracts respiratory features 218 from the wireless reflections and uses them to enhance its stress classification accuracy. The inventors have demonstrated empirically that all three techniques can meaningfully contribute to overall stress classification accuracy.

Body movement feature extraction pipeline 208 can process phase signal 240 to extract body movement features 210 of the user 204. As shown, pipeline 208 can include a displacement power extraction component 242 followed by an intensity/duration component 244. Displacement power extraction component 242 is configured to derive a signal representing power of displacement of phase signal 240 and intensity/duration component 244 can be configured to extract one or more of the following different features based on the displacement power:
(1) Movement intensity: The Movement Intensity (MI) feature is the power of displacement of a unit window (3 min). For a given unit window, component 244 can compute the feature that is the sum of the displacement power between two sample points.

$$M I ( {W}_{i} ) = {\sum }_{n ∈ {W}_{i}} {( ϕ [ n + 1 ] - ϕ [ n ] )}^{2} \qquad ( 2 )$$

where MI(Wᵢ) denotes the movement intensity feature of i^{th} unit window Wᵢ, and ϕ[n] denotes the n^{th} sample point of the extracted phase signal.
(2) Number of high activity occurrences: The number of high activity occurrences represents how often large motion is detected in a unit window.

$$N o H ( {W}_{i} ) = card ( \{ j \vert M I ( {W}_{j} ) > {P}_{th} \} ) \qquad ( 3 )$$

where NoH(Wᵢ) denotes the number of high activity occurrence of i^{th} unit window Wᵢ, card denotes the number of element in a set, Pₜₕ denotes the threshold power for high activity detection (as one example, h=13), and Wⱼ denotes the j^{th} sliding window in a unit window Wᵢ.
(3) Mean intensity of high activity: It represents how large the detected high activities is in a unit window.

$$M i H ( {W}_{i} ) = {\sum }_{M I ( {W}_{j} ) ∈ {S}_{{W}_{i}}} M I ( {W}_{j} ) / card ( {S}_{{W}_{i}} ) \qquad ( 4 )$$

where S_{W}_{ᵢ}={MI(Wⱼ)|MI(Wⱼ)>Pₜₕ}, and where MiH(Wᵢ) denotes the mean intensity of high activity in i^{th} unit window Wᵢ, and S_{W}_{ᵢ} denotes a set of power of j^{th} sliding window Wⱼ in Wᵢ.

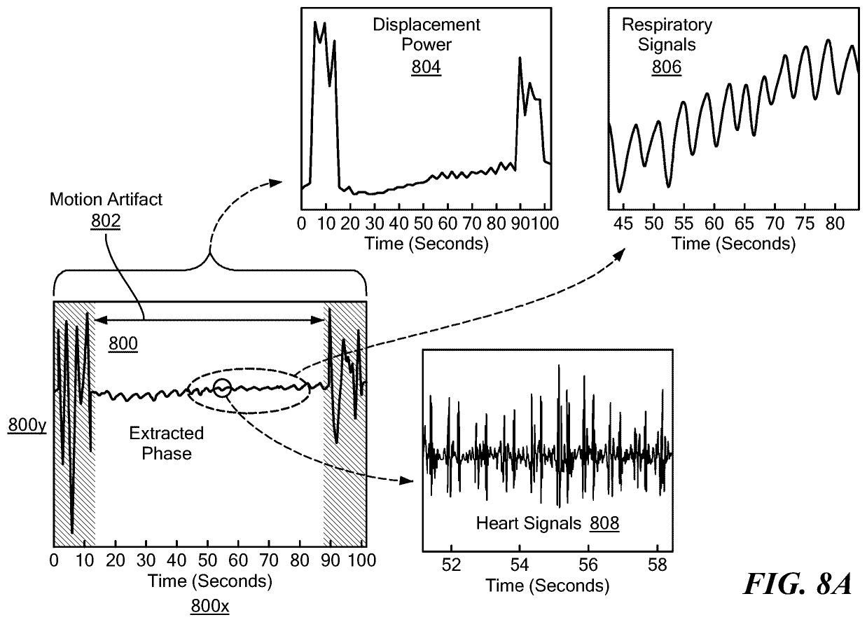

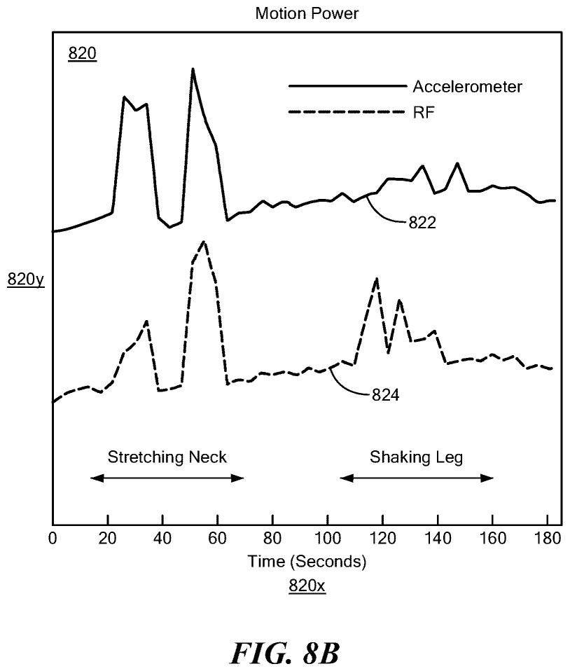

Any or all of the described body movement features 210 can be provided as input to stress classification network 220. The efficacy of feature extraction pipeline 208 has been demonstrated by experimental results described below in the context of FIGS. 8A and 8B. Of note, system 200 is agnostic to the specific type of body movement (e.g., shaking leg or stretching neck). In particular, rather than trying to discover the body movement, system 200 leverages the above movement-agnostic features for use by stress classification network 220.

Respiratory feature extraction pipeline 216 can process phase signal 240 to extract respiratory features 218 of the user 204. To extract respiratory features 218, pipeline 216 can utilize the principal that changes in the speed and depth of respiration are correlated with stress levels as described in the following works: (1) Jennifer A Healey and Rosalind W Picard, 2005, “Detecting stress during real-world driving tasks using physiological sensors,” in “IEEE Transactions on intelligent transportation systems 6, 2 (2005),” 156-166; (2) Yuan Shi, Minh Hoai Nguyen, Patrick Blitz, Brian French, Scott Fisk, Fernando De la Torre, Asim Smailagic, Daniel P Siewiorek, Mustafa al'Absi, Emre Ertin, et al., 2010, “Personalized stress detection from physiological measurements,” in “International symposium on quality of life technology,” 28-29; (3) Chang Zhi Wei., 2013, “Stress emotion recognition based on RSP and EMG signals,” in “Advanced Materials Research, Vol. 709. Trans Tech Publ,” 827-831; and (4) Jacqueline Wijsman, Bernard Grundlehner, Hao Liu, Julien Penders, and Hermie Hermens, 2013, “Wearable physiological sensors reflect mental stress state in office-like situations,” in “2013 Humaine Association Conference on Affective Computing and Intelligent Interaction. IEEE,” 600-605.

Respiratory feature extraction pipeline 216 can implements similar techniques to prior wireless sensing methods for breath monitoring, such as those described in: (1) Fadel Adib, Hongzi Mao, Zachary Kabelac, Dina Katabi, and Robert C Miller, 2015, “Smart homes that monitor breathing and heart rate” in “Proceedings of the 33rd annual ACM conference on human factors in computing systems,” 837-846; and (2) Unsoo Ha, Salah Assana, and Fadel Adib, 2020, “Contactless seismocardiography via deep learning radars,” in “Proceedings of the 26th Annual International Conference on Mobile Computing and Networking,” 1-14.

In particular, as user 204 breathes, their chest expands and contracts, changing the distance from the wireless sensor's 202 antennas and impacting the captured wireless signals, such as shown by the plot 806 of FIG. 8A. Since respiration signals have lower frequency and higher amplitude than those of heart signals, they can be extracted from the captured phase by applying a standard band-pass filter 252, as shown. As one example, filter 252 may be configured with a passband that spans [0.05 0.5 Hz]. In some embodiments, band-pass filter 252 can be applied after identifying the regions without motion artifacts. That is, pipeline 216 may identify the segments in phase signal 240 that are free from motion artifacts, and then pass these segments to bandpass filter 252. Bandpass filter 252 may output a signal referred to herein as a “respiration signal.” Of note, since the signal amplitude of breathing is much larger than heart movement, it is not necessary to reject small motion artifacts from the respiration signal. Bandpass filter 525 can be configured to reject high- and low-frequency noise from the respiration signal. Next, a peak-finding algorithm 254 can be used to identify the local maxima and minima within the respiration signal, which correspond to the inhale and exhale process. As one example, the “findpeaks” function in MATLAB may be used. Subsequently, pipeline 216 can compute the depth (peak-valley) as well as the Mean, SDRR, RMSSD and SDSD of respiration. Any of all of these respiratory features 218 can be provided as input to stress classification network 220.

FIGS. 3-6 illustrate structures and techniques that can be employed by self-similarity-based perception network 248 of FIG. 2A.

Turning to FIGS. 3A-3C, passively extracting the heartbeats from a user's wireless reflection can be challenging. Unlike contact-based physiological biometrics like ECG, which produce a known waveform 300 as depicted in FIG. 3A, heartbeat patterns in wireless signals are generally different each time and are not known a priori. Even within the same experiment and for the same person, this pattern can change. FIG. 3B shows three waveforms 322a, 322b, 322b corresponding to heartbeat patterns of a user during one experiment, captured thirty seconds apart by a wireless sensor (e.g., sensor 202 of FIG. 2). Circular regions 324a, 324b, 324c in the figure highlight that each of respective waveforms 322a, 322b, 322b have their own heartbeat morphology (shape), even though they come from the same user. The reason is that even a small shift in the user's pose during the experiment alters their wireless reflections and changes the captured micro-vibrations arising from their heartbeats.

The second challenge in extracting heartbeats arises from a user's unpredictable body movements. FIG. 3C shows three examples of the captured wireless reflections in everyday scenarios. Waveforms 342a, 342b, 342c correspond to the user eating food, moving back and forth in their chair, and shaking their legs respectively. In more detail, these activities are reflected by artifacts shown in regions 344a, 344b, 344c of their respective waveforms. Consider waveform 342a, where the user is eating food. Different acts while eating such as placing the food in mouth, chewing, and swallowing each give rise to different movements in parts of the body, as illustrated by the artifacts in region 344a. These movements contaminate the captured reflections, easily masking the user's heartbeats. Moreover, these motion artifacts are difficult to filter out since (unlike breathing) they do not occur at a predefined set of frequencies. As highlighted in the other illustrative waveforms 342b, 342c, moving in chair and shaking a leg, each produce their own artifacts (regions 344b, 344c) with different amplitudes.

All of these examples motivate the need for a technique that can (a) reject unpredictable motion artifacts that corrupt the user's wireless reflections, while (b) being able to quickly adapt to the continuously changing morphology of heartbeats in the user's wireless reflections.

Embodiments of the present disclosure overcome the above challenges by exploiting temporally local self-similarities in the captured phase signal. This idea can be understood in the context of FIG. 3B. Notice that in waveforms 322a-322c, although the heartbeat morphology may change over time, each morphology locally repeats multiple times. Thus, this local self-similarity can be exploited in order to identify the heartbeats. In particular, with the goal of extracting the length of individual heartbeats (HRV), disclosed embodiments can search for and identify local self-similarities and use them to extract heartbeat features.

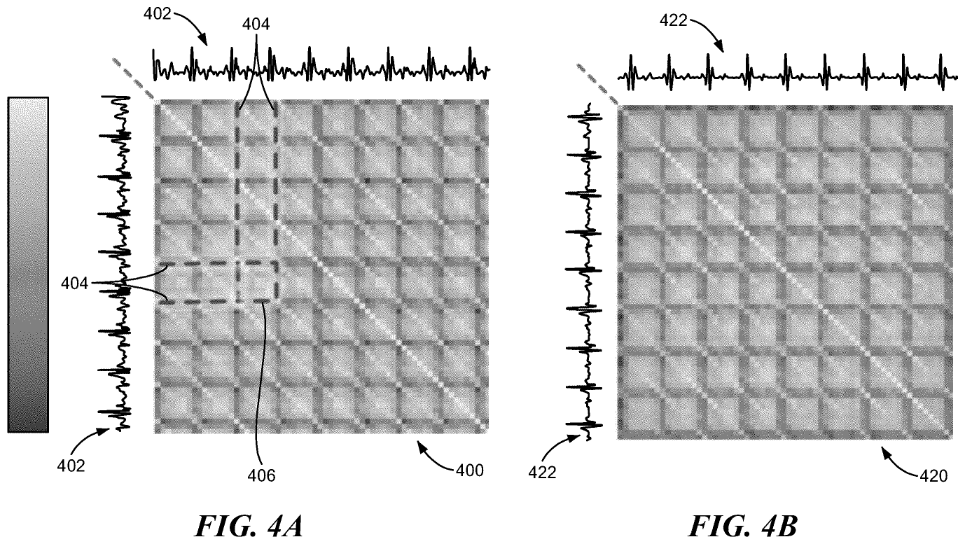

Turning to FIGS. 4A and 4B, to translate this idea into a practical system, a self-similarity matrix (SSM) 400 such as shown in FIG. 4A can be constructed/generated, according to some embodiments. The SSM 400 takes as input two copies of the reflected wireless signal (shown as time-domain signals 402 to the top and rotated to the left-side of the figure) and computes a similarity metric between them. The figure is in the form of a heatmap, where lighter shaded regions indicate high similarity and darker shaded regions indicate low similarity. Consider the two segments highlighted by the horizontal and vertical dashed lines 404. These segments contain very similar patterns. As a result, their corresponding locations in the SSM 400 (the square region 406 at the intersection the four dashed lines) have a high value (lighter shading). Since the entire input signal 402 has heartbeat signals similar to each other, it can be seen that the pattern within square region 406 repeats itself across the SSM 400. Thus, it can be said that the SSM 400 encodes temporally local self-similarities in the signal 402. Thus, systems and techniques disclosed herein use an SSM to extract repeated patterns from relatively short periods of time.

FIG. 4B shows an example of an SSM 420 obtained from another time segment of a captured signal (e.g., a signal responsive to the user's reflections). Similar to FIG. 4A, the input signal in FIG. 4B contains repeated heartbeat patterns, except that the repeated patterns in signal 422 of FIG. 4B are different from those in signal 402 FIG. 4A. Despite their different patterns, the corresponding SSMs 400 and 420 for FIGS. 4A and 4B, respectively, are very similar and capture the periodicity of the repeated signals 402 and 422. This indicates that an SSM does not depend on the particular morphology in the signal, but rather it depends on similarities between those morphologies. Therefore, by looking at morphologies the SSM of the signal, the problem of identifying the shapes of the patterns in the signal can be circumvented, and focus can instead shift to whether those morphologies are repeated.

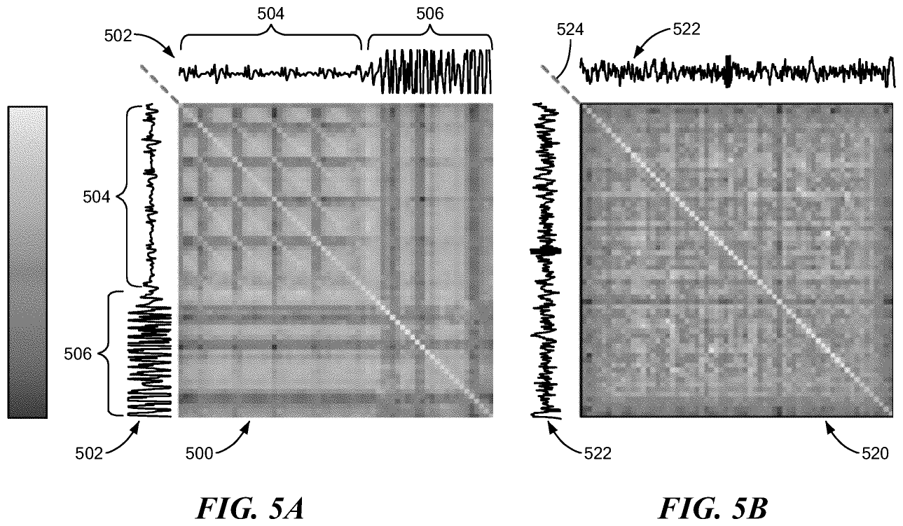

Turning to FIGS. 5A and 5B, embodiments of the present disclosure can utilize SSMs to deal with motion artifacts (e.g., body movements of the user). To demonstrate this, consider a scenario in which a user starts to shake their leg during the measurement, producing a signal 502 having a waveform similar to waveform 342c of FIG. 3C. FIG. 5A shows this signal 502 along with its corresponding SSM 500. Two observations can be made.

First, in the portions of the signal where there are heartbeats (e.g., the portions labeled 504), the corresponding regions in the SSM 500 still show patterns similar to those in FIGS. 4A, 4B.

Second, when the user starts shaking their leg (e.g., within signal portions 506), these patterns disappear in the SSM 500, and a clear pattern is no longer seen, even though it seems shaking leg is a repetitive motion. As discussed below, this can result from the use of a feature extraction network before computing the SSM that only extracts features related to heart signals, and rejects other motions such as shaking legs, even if they include repeating patterns.

The example of FIG. 5A demonstrates an SSM is able to mask out the motion artifacts and noisy parts of the signal while still encoding the similarities between existing heartbeat patterns.

Finally, consider another example of a motion-distorted signal 522, whose SSM 520 is shown in FIG. 5B. In this example, the user is constantly moving their limbs in different directions. Since the user is moving during the entire measurement of signal 522, their heartbeats are completely masked by motion artifacts. Looking at the SSM 520, there are no observable patterns like those that appeared in the previous figures (aside from the lighter shaded region along diagonal 524 which corresponds to the self-correlation). This shows that an SSM may be used to reject regions that do not include any heartbeats, including times when the user is moving so much that the heartbeats are masked, or is not close enough to the device to be detected by the system.

The above discussion demonstrates that by employing an SSM, the systems and techniques disclosed herein can capture local self-similarities in a noisy signal (e.g., a signal generated in response to wireless reflections). Next described is an SSM can be used in order to extract individual heartbeat features.

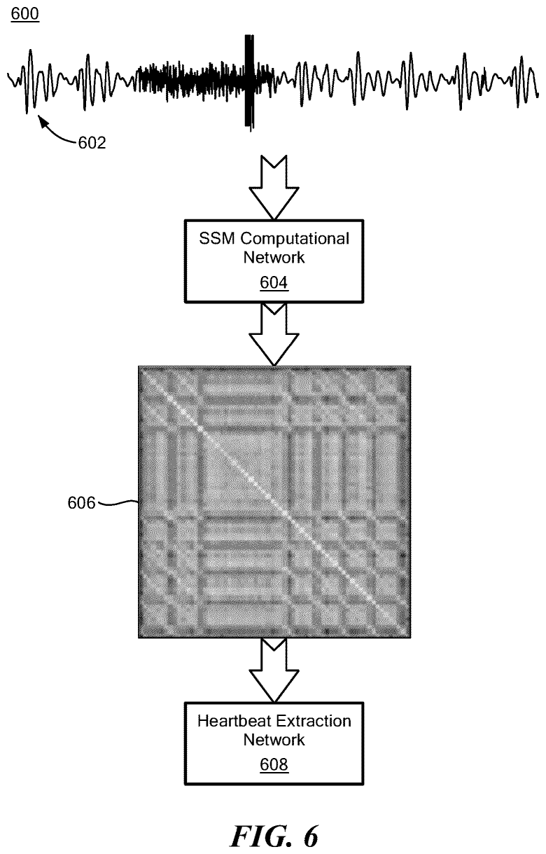

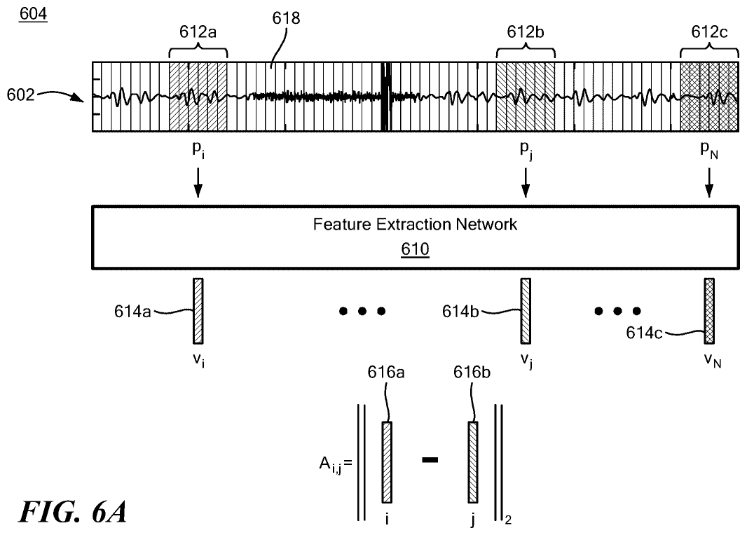

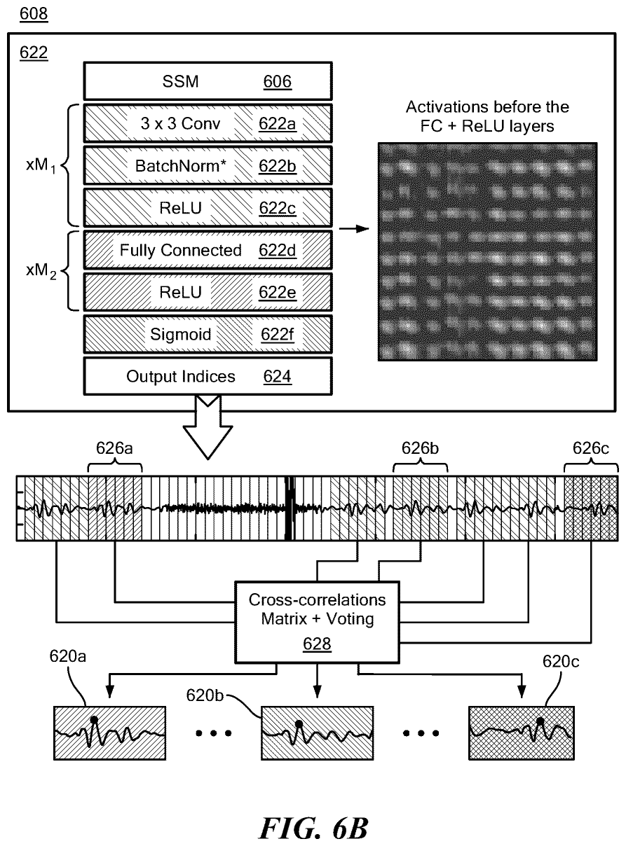

FIG. 6 illustrates a self-similarity-based perception network 600 that can be used to isolate individual heartbeat features. Network 600 can be implemented within and executed by system 120 of FIG. 1B and/or system 200 of FIG. 2. For example, network 600 may be the same as or similar to network 248 of FIG. 2A. The illustrative network 600 consists of two sub-networks: (1) an SSM computation network 604, which takes a signal 602 as input and outputs an SSM 606, and (2) a heartbeat extraction network 608, which takes the computed SSM 606 as input and extracts the precise temporal location of each heartbeat inside the original input signal 602. The input signal 602 may, in general, be a “noisy” signal such as received via wireless reflections from a user's body. FIG. 6A illustrates the SSM computation network 604 in more detail and FIG. 6B illustrates the heartbeat extraction network 608 in more detail. In some embodiments, SSM computation network 604 and/or heartbeat extraction network 608 can include a neural network.

Before describing these two sub-networks 604, 608, a formal definition of a self-similarity matrix is provided. Given a set of segments {p₁, . . . , p_{N}}∈ [symbol] and a similarity function ƒ: [symbol] × [symbol] → [symbol] , the SSM is defined as a N×N matrix A, where A_{i,j}=ƒ(pᵢ, pⱼ).

Turning to FIG. 6A, the first component of network 600 (FIG. 6) is the SSM computation network 604 that computes the SSM 606. To compute the SSM 606, a self-similarity function ƒ is chosen. It is appreciated herein that choosing simple functions like euclidean distance applied directly on the input signal 602 may not capture the similarities between heartbeat patterns well. Instead, SSM computation network 604 is configured to learn this function using a feature extraction network 610.

To start, input signal 602 can be divided into smaller segments, p₁, . . . , p_{N}. Next, each signal segment is independently passed through the feature extraction network 610. FIG. 6A shows the high-level process of passing all the segments through the feature extraction network 610. As shown in the figure, once all feature vectors ν₁, . . . , ν_{N} are computed, the ij^{th} element of the SSM matrix A_{i,j} is computed as the euclidean distance between νᵢ and νⱼ. Hence, the feature extraction network, together with the euclidean distance define the similarity function ƒ.

In some embodiments, feature extraction network 610 can include five (5) layers each with 1, 8, 16, 32 and 64 channels. Each layer has a 1D convolution with rectified linear unit (ReLU) activation and batch norm (BatchNorm). At the end, a global max-pooling can be applied to all channels to get 64 scalars as a feature vector ν1. Note that the max-pooling is performed over the temporal dimension that removes the dependence of the features on time (similar to how the max( ) function returns the same value if the signal is shifted in time). This is particularly important since the objective is to identify similar patterns within each segment, not whether they happen at the end or the beginning of the segment.

Three segments 612a, 612b, and 612c of the input signal 602 are highlighted, illustrating three different heartbeat morphologies that may be exhibited for the same user at different times. Feature vectors 614a, 614b, and 614c may correspond to features extracted from segments 612a, 612b, and 612c, respectively, and columns 616a and 616b of the SSM matrix A_{i,j} may correspond to segments 612a and 612b, respectively.

In designing SSM computation network 604, different lengths and partitions of the input signal 602 may be selected. In some embodiments, two design criteria can be used to help improve the robustness and generalizability of SSM computation network 604. First, the input signal 602 may be segmented in a way to have overlapping regions between adjacent segments. This is because overlapping regions help share information between segments. All the possible starting points for a typical signal are depicted in the figure as vertical lines (e.g., vertical line 618). In some cases, two adjacent starting points may be 100 ms apart. Second, the length of the segments can be chosen such that their duration is less than the minimum heartbeat period but large enough to capture salient features (e.g., peaks or valleys) in the heartbeat morphology. Satisfying these two constraints can help the subsequent heartbeat extraction network 608 in classifying each segment as having zero or one heartbeat. In some embodiments, the segment duration can be selected to be about 400 ms and the resulting overlap between adjacent segments may be about 300 ms.

In some embodiments, SSM computation network 604 can include a neural network.

Turning to FIG. 6B, the second component of network 600 (FIG. 6) is the heartbeat extraction network 608. This network 608 takes the SSM 606 as input, and outputs individual heartbeat intervals 620a, 620b, 620c (620 generally) in the signal. To this end, heartbeat extraction network 608 uses a 2D convolutional neural network 622 that acts directly on the SSM 606. The goal of this neural network 622 is to classify each of the N segments involved in the SSM 606 into one of the two simple groups: ones that contain a heartbeat, and ones that do not contain a heartbeat.

The details of the neural network 622, along with examples of intermediate representations in between layers, are shown in FIG. 6B. In particular, as shown, the neural network 622 can include a 3×3 convolutional layer 622a, a BatchNorm layer 622b, a first ReLU layer 622c, a fully connected layer 622d, a second ReLU layer 622e, and a sigmoid function layer 622f. Layers 622a-622c may be repeated M₁ times (e.g., M₁=6) and layers 622d, 622d can be repeated M₂ times (e.g., M₂⁼2). In some embodiments, a batch size of eight (8) can be used with neural network 622. Herein, “batch size” refers to the number of samples that will be used for a network update, e.g., the number of rows/columns of SSM 606 that are fed into the neural network 622. Such updates can be performed multiple times with multiple batch samples. In some embodiments, the batch size may be selected according to the available computation resources, maximum acceptable training time, and other practical considerations. The illustrated layers 622a-622f are merely illustrative and other arrangements of layers may be used to form neural network 622.

The final output of the neural network 622 is a set of indices 624 indicating which of the patterns that are predicted to be a heartbeat. Here, the indices are reference points for each individual heartbeat, such as a valley or a peak, or a specific feature point. The important thing is that the reference point should have the same criteria for each heartbeat (e.g., 1st peak points for each heartbeat. In the example of FIG. 6B, seven (7) such heartbeat patterns are identified for the given signal 602, with three such segments 626a, 626b, and 626b (626 generally) corresponding to signal segments 612a, 612b, and 612c highlighted in FIG. 6A.

Next, for the group of heartbeat patterns identified by indices 624, heartbeat extraction network 608 is configured to zoom in on each of the coarse time segments 626 to extract fine-grained beat-to-beat intervals 620. To this end, heartbeat extraction network 608 can include a component 628 that utilizes/creates a new, smaller matrix (for example a 7×7 matrix). Row i of the matrix denotes high resolution time-shifts between i and all the other patterns that maximizes their mutual similarity. Therefore, each segment i casts a vote on how much segment j should shift in time to maximize their similarity. Then, for each segment i, component 628 can resolve these votes by taking the median of all the candidate shifts. Finally, by compensating for these mutual time-shifts, component 628 can find the IBIs between consecutive heartbeat patterns. The output heartbeat intervals 620 may are also referred to herein as IBI measurements.

In the example of FIG. 6B, three individual heartbeat intervals 620a, 620b, 620c are shown as output, corresponding to the three signal segments 612a, 612b, and 612c highlighted in FIG. 6.

It is appreciated herein that for the high-resolution heartbeat intervals, the cross-correlation between the raw signal segments (pᵢ) may work better than the extracted features (νᵢ). It is believed that the reason behind this is twofold; first, the feature extraction network 610 (FIG. 6A) includes a global max-pooling over the temporal information which is detrimental to finding the find-grained temporal location of the heartbeat. Second, the similar heartbeat patterns have already been isolated by the preceding network; in other words, if the network is trained properly, the identified patterns should be very similar, like in each of the three waveforms 322a, 322b, 322b in FIG. 3B.

Referring again to FIG. 6, a few additional points are noted. First, one might wonder whether a single end-to-end convolutional network may achieve the same performance as self-similarity-based perception network 600 in extracting individual heartbeats. The reason why such a network would be less effective is that it cannot capture the temporal self-similarities which are reflected in the SSM. Indeed, the inventors have demonstrated through experimentation that prior systems that applied such an approach to extract HRV achieve much lower accuracy than network 600 in the presence of noise and unpredictable movements. Second, before feeding the signal 602 into the SSM computation network 608, a filter (e.g., a differentiator or band-pass filter) can be applied to reject the impact of breathing, which occurs within a predefined frequency range. Such filtering can be performed, for example, by differentiator 246 in FIG. 2A. While a differentiator may generally function a high-pass filter, in practice it may have a stop-band at high frequency region.

Referring back to FIG. 2A, self-similarity-based perception network 248 can output IBI measurements obtained using the techniques and structures described above in the context of FIGS. 6A and 6B. Next described is how heartbeat feature extraction pipeline 212 can select and extract heartbeat features from the IBI measurements and how it can make stress classification network 220 robust to sparsity in the IBI time series due to discarding segments with motion contamination. Since the self-similarity-based perception network 248 is able to extract IBIs, it can compute heartbeat features (or “IBI features”) commonly-used in stress monitoring. These features can be classified into temporal, frequency, and non-linear domains:
(1) Temporal Domain: Mean of IBIs, Standard deviation of IBIs (SDRR), Root Mean Square (RMSSD) and standard deviation (SDSD) of IBIs' successive differences, The percentage of the number of successive IBIs varying more than a given duration (e.g., 50 ms) from the previous interval (i.e., the pNN50 HRV measure may be used)
(2) Frequency Domain: High Frequency Power (HF)
(3) Non-linear Domain: Poincaré analysis (SD2/SD1)

One consideration in selecting heartbeat features is whether all the above features should be used in training its classifier, similar to contact-based stress monitoring methods. Recall that one main difference here is that pipeline 212 needs to discard a significant number of segments due to motion contamination, which can lead to sparsity in IBI time series. However, such sparsity may impact the accuracy of some of the above heartbeat features (which would in turn reduce the overall accuracy of stress classification).

To investigate the effect of sparsity on each of the features, the inventors performed experiments to emulate the discarding of different portion of the IBI time series, and assessed the impact of such sparsity on the accuracy of the each of the computed features. 50-minute IBI series were used from five static subjects. The heartbeat features were extracted from 3-minute segments with a 20-second sliding window. Since motion is continuous, its impact was expected to be on a consecutive chunk of the time series. To simulate such impact, random chunks of different sizes were chosen and removed their IBI estimates from the time series. This simulation was repeated ten times for three different scenarios (discarded percentage: 30%, 50%, 70%) and compared the feature values to those computed using a dense time series (i.e., where 0% of the IBI's were discarded). Mathematically, the error can was computed follows:

$$Error = \frac{\vert Computed Feature ( n \% ) - G round Truth \vert}{G round Truth} \times 100 \qquad ( 5 )$$

where Ground Truth(n %) denotes the feature values when n % of the segment is discarded.

TABLE 1

| IBI Features | Discarded Portion | | |
|---|---|---|---|
| Mean | 1.4% | 1.8% | 2.1% |
| SDRR | 4.8% | 6.8% | 9.3% |
| RMSSD | 3.9% | 5.4% | 8.6% |
| SDSD | 11.1% | 13.8% | 19.3% |
| pNN50 | 3.3% | 8.9% | 13.5% |
| LF | 8.9% | 13.8% | 24.4% |
| HF | 6.2% | 8.9% | 16.6% |
| LF/HF | 11.3% | 23.8% | 31.7% |
| SD2/SD1 | 3.8% | 6.2% | 8.8% |

Table 1 lists the error in computing each of the features as a function of the discarded portion, comparing the error of IBI features as a function of the discarded portion. Interestingly, the temporal and non-linear features are less affected by the discarded regions. In contrast, the frequency features are sensitive to the changes. This is expected because missing data will change the phase of the overall frequency bin and make the frequency resolution worse.

In view of the above experimental results, and according to some embodiments, the LF and LF/HF features may intentionally be excluded for stress classification.

Turning to FIGS. 7A and 7B, it is appreciated herein that it may be necessary to obtain IBI measurements of the user over at least a minimum duration of time (e.g., at least one minute) in order to compute/extract heartbeat features suitable for accurately classifying the user's stress level.

FIG. 7A shows an ideal time series of IBI measurements 700 such as might be output by self-similarity-based perception network 248 of FIG. 2A in response to a heartbeat signal 702 obtained if the user were to remain perfectly still during wireless signal-based stress monitoring. In this idealized scenario, IBI measurements t₁, t₂, . . . , tₙ may be obtained for all of the user's heartbeats during the period Tₛₜₐᵣₜ to T_{end}. Idealized heartbeat features can be computed as:

$${HeartbeatFeat}_{ideal} = f ( {t}_{1} , {t}_{2} , … , {t}_{n} ) , \qquad ( 6 )$$

where f is a function that computes/extracts heartbeat features according to one or more techniques previously described.

FIG. 7B shows a sparse (i.e., non-idealized) time series of IBI measurements 720 that can be output by self-similarity-based perception network 248 of FIG. 2A, in response to a heartbeat signal 722 obtained as the user moves their legs, arms, or other body part during wireless signal-based stress monitoring. In this example, the heartbeats occurring within regions 724a and 724b of signal 722 may be contaminated due to the body motion and the corresponding IBI measurements (e.g., t₃ through t₇ and tₙ₋₄ through tₙ₋₁) may be discarded due to said contamination. In this case, and without further intervention, the heartbeat features may be computed as:

$${HeartbeatFeat}_{contamin ated} = f ( {t}_{1} , {t}_{2} , {t}_{8} , … , {t}_{n - 5} , {t}_{n} ) . \qquad ( 7 )$$

Turning to FIGS. 7C and 7D, and with reference to FIG. 2A, to make stress classification robust to missing/discarded IBI measurements, a sparsity simulation module 740 can be introduced as part of a heartbeat extraction pipeline to augment a sparse time series of IBI measurements. For example, module 740 of FIGS. 7C and 7D may the same as or similar to module 250 of FIG. 2A. The general approach to sparsity simulation is similar to that described above in the simulation for Table 1. Specifically, it reproduces features given the missing IBI assumption and repeats the augmentation one or more times (e.g., ten times) for each window. In more detail, during a training process, original data (i.e., clean signals and ground-truth IBIs) can be augmented in order to make a large number of training data (partially contaminated data from the original one). The total number of training data can be a multiple of the number of original, such as three times, ten times, or more than 10 times. The term “window” used herein refers to a single unit of data from the training data. In some embodiments, IBI measurements may not be used for training if the original series includes excess contamination (e.g., has more than threshold percentage of contamination, such as more than 70%). Using techniques described herein, the stress classification network can learn the patterns and variations of features for real-world measurements.

The illustrative sparsity simulation module 740 includes a trainable ML model 742 that can estimate an ideal time series of IBI measurements from a measured (or “noisy”) heartbeat signal (i.e., a heartbeat signal obtained from wireless reflections and potentially contaminated by body motion). FIG. 7C illustrates how the model 742 can be trained, and FIG. 7D illustrates how the model 742 can be used to estimate an ideal IBI measurements.

Referring to FIG. 7C, during training, an artificial heartbeat signal 744 can be generated by, for example, adding noise to an ideal heartbeat signal (e.g., by artificially adding noise to signal 702 of FIG. 7A). The artificial noise can have random power, distribution, and/or duration. In some embodiments, random noise can be combined with measured motion artifacts, and then added to an ideal heartbeat signal. The artificial heartbeat signal 744 can be fed into a similarity-based perception network (e.g., network 248 of FIG. 2A) to obtain corresponding IBI measurements 746. The combination of the artificial heartbeat signal 744 and the corresponding IBI measurements 746 can then be used to train the model 742. The IBI measurements (which can include, for example, SDRR, RMSSD, and/or SDSD features) may correspond to idealized (i.e., ground truth) IBI measurements that would be expected for the ideal heartbeat signal.

Referring to FIG. 7D, an ideal time series of IBI measurements 752 can be estimated from a measured (noisy) heartbeat signal 748 using the trained model 742. First, the measured heartbeat signal 748 can be fed into a similarity-based perception network (e.g., network 248 of FIG. 2A) to obtain corresponding (sparse) IBI measurements 750. The measured heartbeat signal 748 along with the sparse IBI measurements 750 can then be provided as input to the model 742 to estimate the ideal time series of IBI measurements 752.

FIG. 7E shows an example of a network 760 that can be used in conjunction within the sparsity simulation module of FIGS. 7C and 7D, according to some embodiments. For example, network 760 may be used to train model 742 FIGS. 7C and 7D. The network 760 is similar to that heartbeat extraction network 608 of FIG. 6B, however it differs in that it takes not only an SSM 762 as input, but it also takes an indices matrix 764 as input. Network 760 can be trained using the combined inputs 762, 764. This training can occur separate from that of network 608 but using the same SSM (or a substantially similar SSM). The illustrative network 760 can output IBIs 770, which may be fed into an IBI feature extractor 772 to calculate features such as SDRR, RMSSD, SDSD, etc. using techniques previously discussed.

FIG. 7F shows an example of an indices matrix 780 that can be generated for input to the network of FIG. 7E, according to some embodiments. That is, matrix 780 of FIG. 7F may correspond to matrix 764 of FIG. 7E. As shown, indices matrix 780 can have ones at certain row-and-column positions. The other positions within the matrix can have zeros. The ones positions may correspond to uncontaminated heartbeats (e.g., regions within a measured heartbeat signal not contaminated by motion artifacts), such as those occurring around times Tₛₜₐᵣₜ, Tₛₜₐᵣₜ+1, Tₛₜₐᵣₜ+2, Tₛₜₐᵣₜ+7, Tₛₜₐᵣₜ+8, Tₛₜₐᵣₜ+9, T_{end}−1, and T_{end} in the example of FIG. 7B. All other row-and-column positions may be set to zero in the generated indices matrix 780.

Turning to FIGS. 8A and 8B, next described is an experimental trial demonstrating the three types of features described above, namely body movement features, heartbeat features, and respiratory features. Recall that the disclosed systems and techniques capture the phase of the wireless reflection of the human body. Referring to FIG. 8A, plot 800 shows variations in phase 800y over time 800x during an experiment that lasts for 100 seconds. Notice that in the beginning and the end of the experiment, the phase has significant variations due to motion artifacts 802. This is because for the first and last 15 seconds, the subject waved his hands. FIG. 8A also shows how disclosed systems and techniques can extract three different time-series signals from the phase signal, representing body movements 804, respiration/breathing 806, and heartbeats 808 during the same 100 second period. The figure also shows how disclosed systems and techniques can identify segments when the user is quasi-static (15-85 s) to extract physiological features as well as extract motion-related features from the IBI-discarded regions (0-15 s, 85-100 s). As shown, feature extraction pipelines disclosed herein (e.g., pipelines 208, 212, and 216 of FIG. 2A) can acquire features from three different domains.

Referring to FIG. 8B, to demonstrate that disclosed systems and techniques can sense stress-related motion features, the inventors performed an experiment where the user is asked to perform certain movements. In the experiment, the subject sat about 3 ft away from sensor's antenna. To compare the disclosed systems and techniques to a contact-based sensing approach, an accelerometer was also placed on the user's their chest, with the goal of detecting the body movements. Plot 820 shows resulting computed power of displacement 820y over time 820x from the user-worn accelerometer and from reflected RF signal. Curve 822 represents the computed displacement power from the accelerometer (which is a well-known behavioral feature), and the curve 824 represents the magnitude of displacement computed from the RF reflection. During the experiment, the user stretched his neck (20-70s) and shook his leg (110-160s). As shown, the RF modality can sense the motion in both cases, but the accelerometer cannot detect the leg's movement. Notice that while the accelerometer can detect only the neck motion, the RF modality can sense both motions. This is because the accelerometer placed far from the leg and cannot sense the tiny motion.

Returning to FIG. 2A, having described techniques for extracting body movement features 210, heartbeat features 214, and respiratory features 218 from a noisy time-domain signal 206, the structure and operation of stress classification network 220 is now described. Stress classification network 220 takes the extracted heartbeat features 214 as input, combines them with other features 210, 218 obtained from signal 206, and outputs the user's stress level 222 (e.g., a numeric value indicating stress of the user).

In some embodiments, stress classification network 220 can implement and execute a random forest algorithm for determining a user's stress level 222 using an approach similar to those described in: (1) Yekta Said Can, Niaz Chalabianloo, Deniz Ekiz, and Cem Ersoy, 2019, “Continuous stress detection using wearable sensors in real life: Algorithmic programming contest case study,” “Sensors 19, 8 (2019),” 1849; and (2) Martin Gjoreski, Hristijan Gjoreski, Mitja Luštrek, and Matjaz Gams, 2016, “Continuous stress detection using a wrist device: in laboratory and real life,” in “Proceedings of the 2016 ACM international joint conference on pervasive and ubiquitous computing: Adjunct,” 1185-1193.

The random forest is an ensemble algorithm which learns features by constructing multiple decision trees and has been demonstrated to achieve high accuracy in stress monitoring. In contrast to prior systems and techniques which only relied on heartbeat features, all three of the features 210, 214, 218 described above can be fed into stress classification network 220, according to the present disclosure. The random forest algorithm can select random subsets from the extracted features 210, 214, 218 to create a large number of decision trees. Each decision tree can provide a classification output based on the subsets from the extracted features 210, 214, 218 and the final output (i.e., the user's stress level 222) can be determined by taking the majority vote algorithm from all the decision trees. In some embodiments, the random forest can be implemented using a sklearn library, and the following parameters can be used: n_estimators=500, criterion=“gini”, and max_depth=3.

Next describe are techniques that can be used to train various ML models of system 200. The SSM computation network 604 of FIG. 6A (or, more particularly, feature extraction network 610 thereof) can be trained end-to-end using captured millimeter-wave data and a publicly available SCG dataset, such as that referenced in Miguel A García-González, Ariadna Argelagós-Palau, Mireya Fernández-Chimeno, and Juan Ramos-Castro, 2013, “A comparison of heartbeat detectors for the seismocardiogram, “In Computing in Cardiology 2013, IEEE,” 461-464. In some cases, the “Combined measurement of ECG, Breathing and Seismocardiograms” dataset provided on PhysioNet may be used. The same or similar data can be used train heartbeat extraction network 608 of FIG. 6A (or, more particularly, neural network 622 thereof).

To generate the millimeter-wave data for training, wireless reflections can be captured for one or more test subjects (e.g., at least 4 subjects) and heartbeats can be manually and independently annotated. The collected data can be can then be aggregated and used as ground-truth. In some cases, training data can include around 50 hours of 1D heart recordings. The set of subjects whose heartbeats were used for heartbeat detection training can be disjoint from the set of subjects on which disclosed systems and techniques are evaluated.

In some embodiments, one or more neural networks described herein can be implemented using PyTorch. The cross-entropy loss and an ADAM optimizer with (β₁, β₂)=(0.9, 0.999) can be used to optimize the networks. A learning rate of 1e−4 may be used at the start of training and reduced with a factor of 0.3 whenever the validation loss plateaued for more than five (5) consecutive epochs. During training, a batch size of 8 and set the values M₁=6 and M₂=2 can be used for the heartbeat extraction network 608 (FIG. 6B). The length of the network's input signal can be fixed to be about 6.7 seconds (3550 samples), and the duration of each smaller segment (pᵢ, as discussed in the context of FIG. 6A) can be set to be about 400 milliseconds. The size of the SSM can be set to be 64×64, in some cases.

To help train the described networks and prevent overfitting, a training regime can incorporate a variety of standard data augmentation techniques from the machine learning community. Data augmentation improves the robustness of learning models to noise and to unseen variations in the measurement dataset. In some cases, online data augmentation with 100 epochs (the number of epochs is a hyper-parameter that is fine-tuned based on the validation dataset) can be implemented. In each epoch, every data point (in the context of the self-similarity-based perception network 248, each data point is a 6.7-second time series signal, as described above) is modified prior to feeding it to the network. Below, certain augmentation techniques that can be used are enumerated and the rationale for each of them is explained. Note that whenever the input signal is modified via a certain augmentation technique, the corresponding ground-truth IBI of that signal is updated accordingly.

Time-shifting: This augmentation technique helps the network to be shift invariant, i.e., robust to temporal shifts in the input signal. To implement this technique, the input signal can be shifted by a randomly chosen number of samples between 0 and 500 samples (corresponding to 1 second, i.e., a standard heartbeat).

Linear Time Expansion/Contraction: This augmentation technique helps the network deal with typical variations in inter-beat intervals. To implement it, the input signal can be expanded or contracted time by re-sampling it through a standard anti-aliasing low-pass filter. The expansion range may be randomly chosen between 0.8 and 1.25.

Additive White Gaussian Noise (AWGN): This augmentation technique aims to make the network robust to standard wireless noise. To implement it, AWGN can be added to the signal, with a mean of 0 and a variance randomly chosen between 0.1 and 0.4 of the signal variance.

White Gaussian Noise Replacement: This augmentation technique makes the network robust to erasures in the sensed wireless signal. In the context of wireless stress monitoring described herein, such erasures may arise from sudden and large body motions like standing up. To implement this technique, a random interval inside the signal can be selected and replaced with white noise. Moreover, to represent large movements, the variance of the noise for this augmentation method can be chosen to be 5 to 10 times larger than the signal noise. Of note, here, unlike the additive version, the ground-truth values in the interval are removed. Unlike other augmentations, this augmentation can be applied with a probability of 0.5.

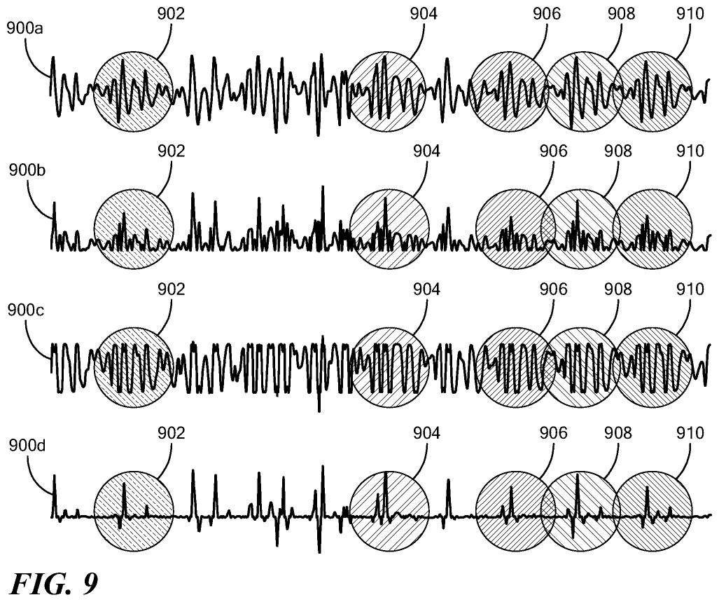

Applying random Polynomials: This augmentation method aims to represent unseen variations in the heartbeat morphology. To implement this augmentation, a non-linear, random polynomial can be applied to all signal samples. Referring to FIG. 9, a first waveform 900a shows a typical wireless signal containing heartbeats, while waveforms 900b, 900c, and 900d are copies of waveform 900a with random polynomials applied. Highlighted regions 902, 904, 906, 908, and 910 in the figure shows the same pattern in different versions of the signal. As can be seen, the original five patterns in waveform 900a have preserved their shape in the other three copies, i.e., waveforms 900b, 900c, and 900d. This shows that while polynomials are a general family of functions, they preserve similar patterns in the signal. Since these patterns are preserved, the network can be trained by augmenting signals, such as the one represented by waveform 900a, to other signals, such as those represented by waveforms 900b, 900c, and 900d, without modifying the ground-truth.

Additional considerations and aspects of the disclosed systems and techniques are now discussed. Certain embodiments described herein may be suitable for performing wireless stress monitoring up to a distance of around 4 meters. This operating range may be extended by incorporating techniques such as beamforming with more antennas or using more sensitive hardware. Certain embodiments are described in terms of monitoring stress for one user at a time. Such embodiments may be extended to accommodate multiple users at the same time through digital beam forming. For example, techniques described in the following may be used to this end: Fadel Adib, Hongzi Mao, Zachary Kabelac, Dina Katabi, and Robert C Miller. 2015, “Smart homes that monitor breathing and heart rate,” in “Proceedings of the 33rd annual ACM conference on human factors in computing systems,” 837-846; and Mingmin Zhao, Yingcheng Liu, Aniruddh Raghu, Tianhong Li, Hang Zhao, Antonio Torralba, and Dina Katabi, 2019, “Through-wall human mesh recovery using radio signals,” in “Proceedings of the IEEE/CVF International Conference on Computer Vision,” 10113-10122. Briefly, and as one example, a wireless sensor can scan the environment and the received wireless signals can be processed to generate 2D images of the subjects, such as illustrated in Fadel Adib, Chen-Yu Hsu, Hongzi Mao, Dina Katabi, and Fredo Durand, 2015, “Capturing the Human Figure Through a Wall,” SIGGRAPH Asia 2015. As another example, heartbeat spectral power can be computed in 3D space and know which direction is optimal as illustrated in Unsoo Ha, Salah Assana, and Fadel Adib, 2020, “Contactless Seismocardiography via Deep Learning Radars,” MobiCom '20, Sep. 21-25, 2020, London, United Kingdom.

While embodiments of the present disclosure are described in terms of processing wireless reflections to extract features of a user in different domains (e.g., respiratory, heartbeats, and body movements), various signal techniques disclosed herein can operate on signals received from non-wireless means. For example, in some embodiments, body movement feature extraction pipeline 208 of FIG. 2 can extract movement features 210 from a signal received from an inertial movement sensor, such as an accelerometer within a wearable device, such as a smartwatch or fitness band. As another example, in some embodiments, a wearable device may output data representing body movements of a user (e.g., arm movements, steps, standing/sitting motion, etc.), and such data may be fed directly into stress classification network 220. As another example, in some embodiments, heartbeat feature extraction pipeline 212 of FIG. 2 can extract heartbeat features from an ECG signal received from an electrode attached to the user or from a user-worn SCG sensor. SCG is a signal modality that can be measured through accelerometers on the chest. e.g., a chest band. As another example, in some embodiments, respiratory feature extraction pipeline 216 of FIG. 2 can extract respiratory features from a PPG signal received from an optical sensor, such as an optical sensor provided within a smartwatch or other wearable device. Any combination of the proceeding signal sources may be used. As yet another example, image data captured by a camera directed at the subject may be processed to extract one or more features of the subject. Various other modalities can be used in conjunction with one or more of the disclosed feature extraction pipelines. For example, heartbeat extraction pipeline 212 can utilize (1) a PPG signal (mainly for heartbeat detection) and a smartwatch signal, or (2) a piezoelectric sensor signal and a chest band signal. As another example, respiratory feature extraction pipeline 216 can utilize (1) an accelerometer sensor signal and a chest band signal, or (2) a piezoelectric sensor signal and a chest band signal.

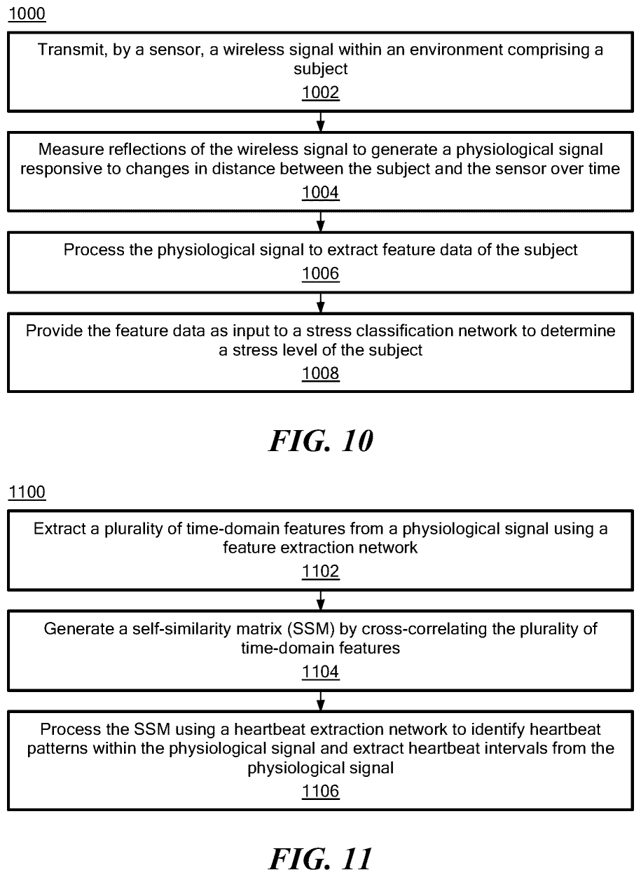

FIGS. 10-12 show illustrative processes for stress monitoring, according to embodiments of the present disclosure. The processes can be implemented within, and executed by, one or more disclosed systems such as system 120 of FIG. 1B and/or system 200 of FIG. 2.

FIG. 10 shows an illustrative process 1000 for measuring stress of a subject using wireless signals. At block 1002, a sensor can transmit a wireless signal within an environment comprising the subject. In some embodiments, the transmitted signal can be a millimeter wave signal. In some embodiments, the transmitted signal can be a Frequency-Modulated Continuous Wave (FMCW) wireless signal. In some embodiments, the sensor can include an antenna array. In some embodiments, the wireless signal can be beamformed in a direction of the subject (e.g., to form a beam directed at a particular subject within an environment having multiple subjects).

At block 1004, reflections of the wireless signal can be measured to generate a physiological signal responsive to changes in distance between the subject and the sensor over time. In some embodiments, this physiological signal can be the same as or similar to phase signal 240 of FIG. 2A, and any of the techniques described above in the context thereof may be used to compute the physiological signal.

At block 1006, the physiological signal can be processed to extract feature data of the subject. The feature can include one or more of: data representing respiration of the subject; data representing heartbeats of the subject; and data representing body movements of the subject. In general, any of the feature extraction techniques described above in the contexts of 2-7 can be utilized here.

In some embodiments, to extract data representing body movements of the subject, block 1006 can include: deriving a signal representing power of displacement of the physiological signal; and extracting one or more different features based on the displacement power, such as movement intensity, number of high activity occurrences, and/or mean intensity of high activity.

In some embodiments, to extract data representing respiration of the subject, block 1006 can include: filtering the physiological signal using a band-pass filter to generate a respiration signal responsive to respiration of the subject; and identifying local maxima and minima of the respiration signal to extract the data representing respiration of the subject.

In some embodiments, to extract data representing heartbeats of the subject, block 1006 can include: dividing the physiological signal into a plurality of time-domain segments; extracting a plurality of time-domain features from the physiological signal by processing individual ones of the plurality of time-domain segments using a feature extraction network; generating an SSM by cross-correlating the plurality of time-domain features; and using the SSM to extract the data representing heartbeats of the subject.

At block 1008, the feature data can be provided as input to a stress classification network to determine a stress level of the subject. For example, a stress classification network similar to network 220 of FIG. 2 can be used to determine the subject's stress level using any of the techniques previously described in conjunction therewith.

FIG. 11 shows an illustrative process 1100 for extracting heartbeats intervals from a noisy time-domain physiological signal. At block 1102, a plurality of time-domain features can be extracted from the physiological signal using a feature extraction network (e.g., network 610 of FIG. 6A). Any of the techniques described above in conjunction with FIG. 6A can be utilized in block 1102.

In some embodiments, block 1102 can include: dividing the physiological signal into a plurality of time-domain segments; and extracting the plurality of time-domain features from the physiological signal by processing individual ones of the plurality of time-domain segments using a feature extraction network.

In some embodiments, process 1100 can include measuring, by a sensor, reflections of a wireless signal to generate the physiological signal responsive to changes in distance between a subject and the sensor over time. In other embodiments, the physiological signal may be received from an electrode. In some embodiments, the physiological signal may be received from a wearable device, such as a smartwatch or fitness band. In some embodiments, physiological signal can correspond to an ECG, PPG, or PPG signal.

At block 1104, an SSM can be generated by cross-correlating the plurality of time-domain features, as represented by matrix A_{i,j} in FIG. 6A.

At block 1106, the SSM can be processed using a heartbeat extraction network (e.g., network 608 of FIG. 6B) to identify heartbeat patterns within the physiological signal and extract the heartbeat intervals from the physiological signal using the identified heartbeat patterns. Any of the techniques described above in conjunction with FIG. 6B can be utilized in block 1106.

In some embodiments, the heartbeat extraction network comprises a CNN (e.g., a two-dimensional CNN). The CNN can be trained to classify individual ones of the plurality of time-domain features as corresponding to a heartbeat or not correspond to a heartbeat. In some embodiments, block 1106 can further include: generating a set of indices indicating which segments of the physiological signal correspond to heartbeats based on the classifications, wherein the heartbeat extraction network extracts the heartbeat intervals using the set of indices.

In some cases, the extracted heartbeat intervals (e.g., IBIs) may be relatively sparse over time due to body movement contamination. Thus, in some embodiments, a sparsity simulation (such as described above in the context of FIGS. 7A-7D) may be used to generate an estimate of an “ideal” IBI time series.

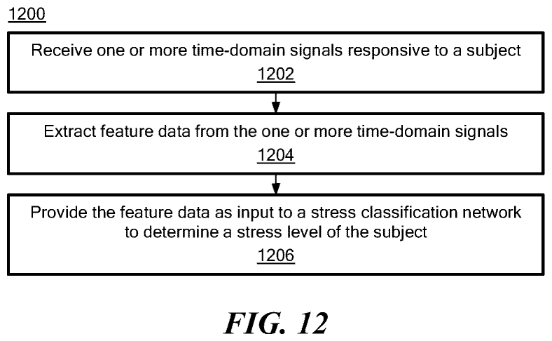

FIG. 12 shows an illustrative process 1200 for measuring stress of a subject. At block 1202, one or more time-domain signals responsive to the subject can be received. In some embodiments, at least one of the one or more time-domain signals can include a physiological signal and block 1202 can include measuring, by a sensor, reflections of a wireless signal to generate the physiological signal responsive to changes in distance between the subject and the sensor over time. In some embodiments, at least one of the one or more time-domain signals can be received from a wearable device associated with the subject. In some embodiments, at least one of the one or more time-domain signals can be received from an electrode associated with the subject. In some embodiments, at least one of the one or more time-domain signals can be received from a camera directed at the subject

At block 1204, feature data can be extracted from the one or more time-domain signals. The feature data may include, for example, data representing vital signs of the subject (e.g., heartbeats and respiration), as well as data representing body movements of the subject.

At block 1206, the feature data can be provided as input to a stress classification network (e.g., network 220 of FIG. 2) to determine a stress level of the subject. In some embodiments, 30. In some embodiments, the stress classification network may be trained using datasets of time-domain signals from subjects under stress. In some embodiments, the stress classification network may include a neural network. In some embodiments, the stress classification network may include a random forest classifier.

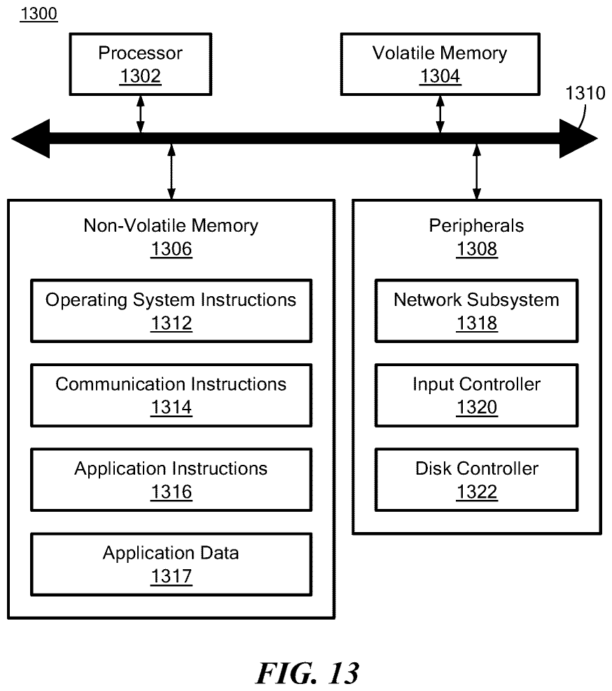

FIG. 13 shows an illustrative server device 1300 that may implement various features and processes as described herein (e.g., the processing described above in the context of FIGS. 4 and 4A). The server device 1300 may be implemented on any electronic device that runs software applications derived from compiled instructions, including without limitation personal computers, servers, smart phones, media players, electronic tablets, game consoles, email devices, etc. In some implementations, the server device 1300 may include one or more processors 1302, volatile memory 1304, non-volatile memory 1306, and one or more peripherals 1308. These components may be interconnected by one or more computer buses 1310.

Processor(s) 1302 may use any known processor technology, including but not limited to graphics processors and multi-core processors. Suitable processors for the execution of a program of instructions may include, by way of example, both general and special purpose microprocessors, and the sole processor or one of multiple processors or cores, of any kind of computer. Bus 1310 may be any known internal or external bus technology, including but not limited to ISA, EISA, PCI, PCI Express, NuBus, USB, Serial ATA or FireWire. Volatile memory 1304 may include, for example, SDRAM. Processor 1302 may receive instructions and data from a read-only memory or a random access memory or both. The essential elements of a computer may include a processor for executing instructions and one or more memories for storing instructions and data.

Non-volatile memory 1306 may include by way of example semiconductor memory devices, such as EPROM, EEPROM, and flash memory devices; magnetic disks such as internal hard disks and removable disks; magneto-optical disks; and CD-ROM and DVD-ROM disks. Non-volatile memory 1306 may store various computer instructions including operating system instructions 1312, communication instructions 1314, application instructions 1316, and application data 1317. Operating system instructions 1312 may include instructions for implementing an operating system (e.g., Mac OS®, Windows®, or Linux). The operating system may be multi-user, multiprocessing, multitasking, multithreading, real-time, and the like. Communication instructions 1314 may include network communications instructions, for example, software for implementing communication protocols, such as TCP/IP, HTTP, Ethernet, telephony, etc.

Peripherals 1308 may be included within the server device 1300 or operatively coupled to communicate with the server device 1300. Peripherals 1308 may include, for example, network interfaces 1318, input devices 1320, and storage devices 1322. Network interfaces may include for example an Ethernet or Wi-Fi adapter. Input devices 1320 may be any known input device technology, including but not limited to a keyboard (including a virtual keyboard), mouse, trackball, and touch-sensitive pad or display. Storage devices 1322 may include one or more mass storage devices for storing data files; such devices include magnetic disks, such as internal hard disks and removable disks; magneto-optical disks; and optical disks.

The system can perform processing, at least in part, via a computer program product, (e.g., in a machine-readable storage device), for execution by, or to control the operation of, data processing apparatus (e.g., a programmable processor, a computer, or multiple computers). Each such program may be implemented in a high-level procedural or object-oriented programming language to communicate with a computer system. However, the programs may be implemented in assembly or machine language. The language may be a compiled or an interpreted language and it may be deployed in any form, including as a stand-alone program or as a module, component, subroutine, or other unit suitable for use in a computing environment. A computer program may be deployed to be executed on one computer or on multiple computers at one site or distributed across multiple sites and interconnected by a communication network. A computer program may be stored on a storage medium or device (e.g., CD-ROM, hard disk, or magnetic diskette) that is readable by a general or special purpose programmable computer for configuring and operating the computer when the storage medium or device is read by the computer. Processing may also be implemented as a machine-readable storage medium, configured with a computer program, where upon execution, instructions in the computer program cause the computer to operate. The program logic may be run on a physical or virtual processor. The program logic may be run across one or more physical or virtual processors.

In illustrative implementations of the concepts described herein, one or more computers (e.g., integrated circuits, microcontrollers, controllers, microprocessors, processors, field-programmable-gate arrays, personal computers, onboard computers, remote computers, servers, network hosts, or client computers) may be programmed and specially adapted: (1) to perform any computation, calculation, program or algorithm described or implied above; (2) to receive signals indicative of human input; (3) to output signals for controlling transducers for outputting information in human perceivable format; (4) to process data, to perform computations, to execute any algorithm or software, and (5) to control the read or write of data to and from memory devices. The one or more computers may be connected to each other or to other components in the system either: (a) wirelessly, (b) by wired or fiber optic connection, or (c) by any combination of wired, fiber optic or wireless connections.

In illustrative implementations of the concepts described herein, one or more computers may be programmed to perform any and all computations, calculations, programs and algorithms described or implied above, and any and all functions described in the immediately preceding paragraph. Likewise, in illustrative implementations of the concepts described herein, one or more non-transitory, machine-accessible media may have instructions encoded thereon for one or more computers to perform any and all computations, calculations, programs and algorithms described or implied above, and any and all functions described in the immediately preceding paragraph.

For example, in some cases: (a) a machine-accessible medium may have instructions encoded thereon that specify steps in a software program; and (b) the computer may access the instructions encoded on the machine-accessible medium, in order to determine steps to execute in the software program. In illustrative implementations, the machine-accessible medium may comprise a tangible non-transitory medium. In some cases, the machine-accessible medium may comprise (a) a memory unit or (b) an auxiliary memory storage device. For example, in some cases, while a program is executing, a control unit in a computer may fetch the next coded instruction from memory.

In some cases, one or more computers are programmed for communication over a network. For example, in some cases, one or more computers are programmed for network communication: (a) in accordance with the Internet Protocol Suite, or (b) in accordance with any other industry standard for communication, including any USB standard, ethernet standard (e.g., IEEE 802.3), token ring standard (e.g., IEEE 802.5), or wireless communication standard, including IEEE 802.11 (Wi-Fi®), IEEE 802.15 (Bluetooth®/Zigbee®), IEEE 802.16, IEEE 802.20, GSM (global system for mobile communications), UMTS (universal mobile telecommunication system), CDMA (code division multiple access, including IS-95, IS-2000, and WCDMA), LTE (long term evolution), or 5G (e.g., ITU IMT-2020).

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter.

Accordingly, although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

Subject matter described herein can be implemented in digital electronic circuitry, or in computer software, firmware, or hardware, including the structural means disclosed herein and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or another unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this disclosure, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., a field-programmable gate array (FPGA) or an ASIC.

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of nonvolatile memory, including by ways of example semiconductor memory devices, such as EPROM, EEPROM, flash memory device, or magnetic disks. The processor and the memory can be supplemented by, or incorporated in, special purpose logic circuitry.

In the foregoing detailed description, various features are grouped together in one or more individual embodiments for the purpose of streamlining the disclosure. This method of disclosure is not to be interpreted as reflecting an intention that each claim requires more features than are expressly recited therein. Rather, inventive aspects may lie in less than all features of each disclosed embodiment.

As used herein, the terms “comprises,” “comprising,” “includes,” “including,” “has,” “having,” “contains” or “containing,” or any other variation thereof, are intended to cover a nonexclusive inclusion. For example, system, method, or apparatus that comprises a list of elements is not necessarily limited to only those elements but can include other elements not expressly listed or inherent to such composition, mixture, process, method, article, or apparatus.

The term “one or more” is understood to include any integer number greater than or equal to one, i.e. one, two, three, four, etc. The terms “a plurality” is understood to include any integer number greater than or equal to two, i.e. two, three, four, five, etc.

References in the specification to “one embodiment,” “an embodiment,” “an example embodiment,” etc., indicate that the embodiment described can include a particular feature, structure, or characteristic, but every embodiment can include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Use of ordinal terms such as “first,” “second,” “third,” etc., in the specification to modify an element does not by itself connote any priority, precedence, or order of one element over another or the temporal order in which acts of a method are performed, but are used merely as labels to distinguish one element having a certain name from another element having a same name (but for use of the ordinal term) to distinguish the elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments. The term “substantially” may be used to refer to values that are within ±20% of a comparative measure in some embodiments, within ±10% in some embodiments, within ±5% in some embodiments, and yet within ±2% in some embodiments.

The disclosed subject matter is not limited in its application to the details of construction and to the arrangements of the components set forth in the following description or illustrated in the drawings. The disclosed subject matter is capable of other embodiments and of being practiced and carried out in various ways. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

All publications and references cited herein are expressly incorporated herein by reference in their entirety.

1. A method for measuring stress of a subject, the method comprising:
transmitting, by a sensor, a wireless signal within an environment comprising the subject;
measuring reflections of the wireless signal to generate a physiological signal responsive to changes in distance between the subject and the sensor over time;
processing the physiological signal to extract feature data of the subject; and
providing the feature data as input to a stress classification network to determine a stress level of the subject.

2. The method of claim 1, wherein the feature data comprises data representing respiration of the subject.

3. The method of claim 2, wherein the processing of the physiological signal comprises:
filtering the physiological signal using a band-pass filter to generate a respiration signal responsive to respiration of the subject; and
identifying local maxima and minima of the respiration signal to extract the data representing respiration of the subject.

4. The method of claim 1, wherein the feature data comprises data representing heartbeats of the subject.

5. The method of claim 4, wherein the processing of the physiological signal comprises:
dividing the physiological signal into a plurality of time-domain segments;
extracting a plurality of time-domain features from the physiological signal by processing individual ones of the plurality of time-domain segments using a feature extraction network;
generating a self-similarity matrix (SSM) by cross-correlating the plurality of time-domain features; and
using the SSM to extract the data representing heartbeats of the subject.

6. The method of claim 1, wherein the feature data comprises data representing body movements of the subject, said movements being associated with respiration and/or heartbeat of the subject.

7. (canceled)

8. The method of claim 1, wherein the transmitting of the wireless signal comprises transmitting at least one of a millimeter wave signal and a Frequency-Modulated Continuous Wave (FMCW) wireless signal.

9-10. (canceled)

11. The method of claim 1, wherein the transmitting of the wireless signal comprises transmitting the wireless signal via an antenna array of the sensor and the environment comprises multiple subjects, the method further comprising beamforming the wireless signal in a direction of the subject.

12. A method for extracting heartbeats intervals from a noisy time-domain physiological signal, the method comprising:
extracting a plurality of time-domain features from the physiological signal using a feature extraction network;
generating a self-similarity matrix (SSM) by cross-correlating the plurality of time-domain features;
processing the SSM using a heartbeat extraction network to:
identify heartbeat patterns within the physiological signal; and
extract the heartbeat intervals using the identified heartbeat patterns.

13. The method of claim 12, further comprising:
measuring, by a sensor, reflections of a wireless signal to generate the physiological signal responsive to changes in distance between a subject and the sensor over time.

14. The method of claim 12, wherein the physiological signal is received from at least one of a wireless reflection, an electrode, and a wearable device.

15. (canceled)

16. The method of claim 12, wherein the physiological signal corresponds to at least one of an electrocardiogram (ECG) signal, a photoplethysmography (PPG) signal, and a seismocardiograph (SCG) signal.

17-18. (canceled)

19. The method of claim 12, wherein the extracting of the plurality of time-domain features from the physiological signal comprises:
dividing the physiological signal into a plurality of time-domain segments; and
extracting the plurality of time-domain features from the physiological signal by processing individual ones of the plurality of time-domain segments using a feature extraction network.

20. The method of claim 12, wherein the heartbeat extraction network comprises a two-dimensional (2D) convolutional neural network (CNN) trained to classify individual ones of the plurality of time-domain features as corresponding to a heartbeat or not corresponding to a heartbeat.

21. (canceled)

22. The method of claim 20, further comprising:
generating a set of indices indicating which segments of the physiological signal correspond to heartbeats based on the classifications,
wherein the heartbeat extraction network extracts the heartbeat intervals using the set of indices.

23. A method for measuring stress of a subject, comprising:
receiving one or more time-domain signals responsive to the subject;
extracting feature data from the one or more time-domain signals, the feature data including at least:
data representing vital signs of the subject, and
data representing body movements of the subject; and
providing the feature data as input to a stress classification network to determine a stress level of the subject.

24. The method of claim 23, wherein the receiving of the one or more time-domain signals includes receiving a physiological signal, the method further comprising:
measuring, by a sensor, reflections of a wireless signal to generate the physiological signal responsive to changes in distance between the subject and the sensor over time.

25. The method of claim 24, wherein the receiving of the one or more time-domain signals includes receiving a signal from at least one of a wearable device associated with the subject, an electrode associated with the subject, a camera directed at the subject.

26-27. (canceled)

28. The method of claim 23, wherein the data representing vital signs of the subject includes at least one of data representing respiration of the subject and data representing heartbeats of the subject.

29. (canceled)

30. The method of claim 23, wherein the stress classification network is trained using datasets of time-domain signals from subjects under stress.

31-32. (canceled)
